SEQUENCE LISTING (23 sequences)

SEQ ID NO: 1 - protein, 481 aa, Homo sapiens [sequence omitted]

SEQ ID NO: 2 - protein, 481 aa, Homo sapiens
MYDAERGWSLSFAGCGFLGFYHVGATRCLSEHAPHLLRDARMLFGASAGALHCVGVLSGIPLEQTLQVLSDLVRKARSRNIGIFHPSFNLSKFLRQGLCKCLPANVHQLISGKIGISLTRVSDGENVLVSDFRSKDEVVDALVCSCFIPFYSGLIPPSFRGVRYVDGGVSDNVPFIDAKTTITVSPFYGEYDICPKVKSTNFLHVDITKLSLRLCTGNLYLLSRAFVPPDLKVLGEICLRGYLDAFRFLEEKGICNRPQPGLKSSSEGMDPEVAMPSWANMSLDSSPESAALAVRLEGDELLDHLRLSILPWDESILDTLSPRLATALSEEMKDKGGYMSKICNLLPIRIMSYVMLPCTLPVESAIAIVQRLVTWLPDMPDDVLWLQWVTSQVFTRVLMCLLPASRSQMPVSSQQASPCTPEQDWPCWTPCSPKGCPAETKAEATPRSILRSSLNFFLGNKVPAGAEGLSTFPSFSLEKSL

SEQ ID NO: 3 - DNA, 1446 bp, Homo sapiens
  CDS (1)..(1443)
atgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccggtatcccgctggagcagactctgcaggtcctctcagatcttgtgcggaaggccaggagtcggaacattggcatcttccatccatccttcaacttaagcaagttcctccgacagggtctctgcaaatgcctcccggccaatgtccaccagctcatctccggcaaaataggcatctctcttaccagagtgtctgatggggaaaacgttctggtgtctgactttcggtccaaagacgaagtcgtggatgccttggtatgttcctgcttcatccccttctacagtggccttatccctccttccttcagaggcgtgcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgctgggagagatatgccttcgaggatatttggatgcattcaggttcttggaagagaagggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacagcactgagtgaagaaatgaaagacaaaggtggatacatgagcaagatttgcaacttgctacccattaggataatgtcttatgtaatgctgccctgtaccctgcctgtggaatctgccattgcgattgtccagagactggtgacatggcttccagatatgcccgacgatgtcctgtggttgcagtgggtgacctcacaggtgttcactcgagtgctgatgtgtctgctccccgcctccaggtcccaaatgccagtgagcagccaacaggcctccccatgcacacctgagcaggactggccctgctggactccctgctcccccgagggctgtccagcagagaccaaagcagaggccaccccgcggtccatcctcaggtccagcctgaacttcttcttgggcaataaagtacctgctggtgctgaggggctctccacctttcccagtttttcactagagaagagtctgtga

SEQ ID NO: 4 - DNA, 1446 bp, Homo sapiens
  CDS (1)..(1443)
atgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccggtatcccgctggagcagactctgcaggtcctctcagatcttgtgcggaaggccaggagtcggaacattggcatcttccatccatccttcaacttaagcaagttcctccgacagggtctctgcaaatgcctcccggccaatgtccaccagctcatctccggcaaaataggcatctctcttaccagagtgtctgatggggaaaacgttctggtgtctgactttcggtccaaagacgaagtcgtggatgccttggtatgttcctgcttcatccccttctacagtggccttatccctccttccttcagaggcgtgcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgctgggagagatatgccttcgaggatatttggatgcattcaggttcttggaagagaagggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacagcactgagtgaagaaatgaaagacaaaggtggatacatgagcaagatttgcaacttgctacccattaggataatgtcttatgtaatgctgccctgtaccctgcctgtggaatctgccattgcgattgtccagagactggtgacatggcttccagatatgcccgacgatgtcctgtggttgcagtgggtgacctcacaggtgttcactcgagtgctgatgtgtctgctccccgcctccaggtcccaaatgccagtgagcagccaacaggcctccccatgcacacctgagcaggactggccctgctggactccctgctcccccaagggctgtccagcagagaccaaagcagaggccaccccgcggtccatcctcaggtccagcctgaacttcttcttgggcaataaagtacctgctggtgctgaggggctctccacctttcccagtttttcactagagaagagtctgtga

SEQ ID NO: 5 - DNA, 762 bp, Homo sapiens
  CDS (1)..(759)
atgaagcacatcaacctatcatttgcagcgtgtggatttctgggcatttaccacttgggggcagcatctgcactttgcagacatggcaaaaaacttgtgaaggatgtcaaagccttcgctggggcgtctgcgggatcgttggttgcttctgttctgctaacagcaccagaaaaaatagaggaatgtaaccaatttacctacaagtttgccgaagaaatcagaaggcagtctttcggggcagtaacgcccggttatgacttcatggcccgactaagaagtgggatggagtcgattcttcctcccagcgctcacgagctggcccagaaccgactgcacgtatccatcaccaacgccaaaaccagagaaaatcacttagtctccactttttcctccagggaggacctcattaaggtcctcctagccagcagttttgtgcccatttatgcaggactgaagctagtggaatacaaagggcagaagtgggtggacggaggcctcaccaacgctcttcccatcctgcccgtcggccggacagtaaccatctcccccttcagtggacgactggacatctccccgcaggacaaagggcagctagatctgtatgttaatatcgccaagcaggatatcatgttgtccctggcaaacctggtgagactcaaccaagccctttttcccccaagcaagaggaaaatggaatctttgtatcagtgtggttttgatgacactgttaagtttttacttaaagaaaattggtttgaataa

SEQ ID NO: 6 - protein, 253 aa, Homo sapiens
MKHINLSFAACGFLGIYHLGAASALCRHGKKLVKDVKAFAGASAGSLVASVLLTAPEKIEECNQFTYKFAEEIRRQSFGAVTPGYDFMARLRSGMESILPPSAHELAQNRLHVSITNAKTRENHLVSTFSSREDLIKVLLASSFVPIYAGLKLVEYKGQKWVDGGLTNALPILPVGRTVTISPFSGRLDISPQDKGQLDLYVNIAKQDIMLSLANLVRLNQALFPPSKRKMESLYQCGFDDTVKFLLKENWFE

SEQ ID NO: 7 - DNA, 1515 bp, Homo sapiens
  CDS (1)..(1512)
atgtttccccgcgagaagacgtggaacatctcgttcgcgggctgcggcttcctcggcgtctactacgtcggcgtggcctcctgcctccgcgagcacgcgcccttcctggtggccaacgccacgcacatctacggcgcctcggccggggcgctcacggccacggcgctggtcaccggggtctgcctgggtgaggctggtgccaagttcattgaggtatctaaagaggcccggaagcggttcctgggccccctgcacccctccttcaacctggtaaagatcatccgcagtttcctgctgaaggtcctgcctgctgatagccatgagcatgccagtgggcgcctgggcatctccctgacccgcgtgtcagacggcgagaatgtcattatatcccacttcaactccaaggacgagctcatccaggccaatgtctgcagcggtttcatccccgtgtactgtgggctcatccctccctccctccagggggtgcgctacgtggatggtggcatttcagacaacctgccactctatgagcttaagaacaccatcacagtgtcccccttctcgggcgagagtgacatctgtccgcaggacagctccaccaacatccacgagctgcgggtcaccaacaccagcatccagttcaacctgcgcaacctctaccgcctctccaaggccctcttcccgccggagcccctggtgctgcgagagatgtgcaagcagggataccgggatggcctgcgctttctgcagcggaacggcctcctgaaccggcccaaccccttgctggcgttgccccccgcccgcccccacggcccagaggacaaggaccaggcagtggagagcgcccaagcggaggattactcgcagctgcccggagaagatcacatcctggagcacctgcccgcccggctcaatgaggccctgctggaggcctgcgtggagcccacggacctgctgaccaccctctccaacatgctgcctgtgcgtctggccacggccatgatggtgccctacacgctgccgctggagagcgctctgtccttcaccatccgcttgctggagtggctgcccgacgttcccgaggacatccggtggatgaaggagcagacgggcagcatctgccagtacctggtgatgcgcgccaagaggaagctgggcaggcacctgccctccaggctgccggagcaggtggagctgcgccgcgtccagtcgctgccgtccgtgccgctgtcctgcgccgcctacagagaggcactgcccggctggatgcgcaacaacctctcgctgggggacgcgctggccaagtgggaggagtgccagcgccagctgctgctcggcctcttctgcaccaacgtggccttcccgcccgaagctctgcgcatgcgcgcacccgccgacccggctcccgcccccgcggacccagcatccccgcagcaccagccggccgggcctgcccccttgctgagcacccctgctcccgaggcccggcccgtgatcggggccctggggctgtga

SEQ ID NO: 8 - protein, 504 aa, Homo sapiens [sequence omitted]

SEQ ID NO: 9 - protein, 482 aa, Homo sapiens
MYDAERGWSLSFAGCGFLGFYHVGATRCLSEHAPHLLRDARMLFGASAGALHCVGVLSGIPLEQTLQVLSDLVRKARSRNIGIFHPSFNLSKFLRQGLCKCLPANVHQLISGKIGISLTRVSDGENVLVSDFRSKDEVVDALVCSCFIPFYSGLIPPSFRGVRYVDGGVSDNVPFIDAKTTITVSPFYGEYDICPKVKSTNFLHVDITKLSLRLCTGNLYLLSRAFVPPDLKVLGEICLRGYLDAFRFLEEKGICNRPQPGLKSSSEGMDPEVAMPSWANMSLDSSPESAALAVRLEGDELLDHLRLSILPWDESILDTLSPRLATALSEEMKDKGGYMSKICNLLPIRIMSYVMLPCTLPVESAIAIVQRLVTWLPDMPDDVLWLQWVTSQVFTRVLMCLLPASRSQMPVSSQQASPCTPEQDWPCWTPCSPEGCPAETKAEATPRSILRSSLNFFLGNKVPAGAEGLSTFPSFSLEKSLH

SEQ ID NO: 10 - protein, 505 aa, Homo sapiens
MFPREKTWNISFAGCGFLGVYYVGVASCLREHAPFLVANATHIYGASAGALTATALVTGVCLGEAGAKFIEVSKEARKRFLGPLHPSFNLVKIIRSFLLKVLPADSHEHASGRLGISLTRVSDGENVIISHFNSKDELIQANVCSGFIPVYCGLIPPSLQGVRYVDGGISDNLPLYELKNTITVSPFSGESDICPQDSSTNIHELRVTNTSIQFNLRNLYRLSKALFPPEPLVLREMCKQGYRDGLRFLQRNGLLNRPNPLLALPPARPHGPEDKDQAVESAQAEDYSQLPGEDHILEHLPARLNEALLEACVEPTDLLTTLSNMLPVRLATAMMVPYTLPLESALSFTIRLLEWLPDVPRDIRWMKEQTGSICQYLVMRAKRKLGRHLPSRLPEQVELRRVQSLPSVPLSCAAYREALPGWMRNNLSLGDALAKWEECQRQLLLGLFCTNVAFPPEALRMRAPADPAPAPADPASPQHQPAGPAPLLSTPAPEARPVIGALGLH

SEQ ID NO: 11 - protein, 262 aa, Homo sapiens
MKHINLSFAACGFLGIYHLGAASALCRHGKKLVKDVKAFAGASAGFTSLVASVLLTAPEKIEECNQFTYKFAEEIRRQSFGAVTPGYDFMARLRSGMESILPPSAHFTELAQNRLHVSITNAKTRENHLVSTFSSREDLIKVLLASSFVPIYAGLKLVEYKGQKWVDFTGGLTNALPILPVGRTVTISPFSGRLDISPQDKGQLDLYVNIAKQDIMLSLANLVRLNQAFTLFPPSKRKMESLYQCGFDDTVKFLLKENWFEH

SEQ ID NO: 12 - protein, 44 aa, Solanum tuberosum
GGGIKGIIPAIILEFLEGQLQEVDNNKDARLADYFDVIGGTSTG

SEQ ID NO: 13 - protein, 37 aa, Mus sp.
GGGVKGLVIIQLLIAIEKASGVATKDLFDWVAGTSTG

SEQ ID NO: 14 - protein, 37 aa, Homo sapiens
GGGTRGVVALQTLRKLVELTQKPVHQLFDYICGVSTG

SEQ ID NO: 15 - protein, 36 aa, Homo sapiens
GCGFLGFYHVGATRCLSEHAPHLLRDARMLFGASAG

SEQ ID NO: 16 - protein, 9 aa, Mus sp.
KVKTVPLHL

SEQ ID NO: 17 - DNA, 20 bp, Artificial Sequence
  Description of Artificial Sequence Synthetic oligonucleotide
ggatggtagtccggagaaca

SEQ ID NO: 18 - DNA, 23 bp, Artificial Sequence
  Description of Artificial Sequence Synthetic oligonucleotide
agtctggatcgttcagaatcaag

SEQ ID NO: 19 - DNA, 22 bp, Artificial Sequence
  Description of Artificial Sequence Synthetic oligonucleotide
tctcgatttctcgacggcgccg

SEQ ID NO: 20 - DNA, 24 bp, Artificial Sequence
  Description of Artificial Sequence Synthetic oligonucleotide
tgaagaaaatgggtttctcgtcat

SEQ ID NO: 21 - DNA, 20 bp, Artificial Sequence
  Description of Artificial Sequence Synthetic oligonucleotide
acgatccctggtggtttcac

SEQ ID NO: 22 - DNA, 20 bp, Artificial Sequence
  Description of Artificial Sequence Synthetic oligonucleotide
actctgctcgaaaacgttga

SEQ ID NO: 23 - protein, 4 aa, Mus sp.
KKVS

(19) United States
(12) Patent Application Publication
(10) Pub. No.: US 2005/0214275 A1
(43) Pub. Date: Sep. 29, 2005

(54) Sequential ordered fatty acid alpha oxidation and delta9 desaturation are major determinants of lipid storage and utilization in adipocytes

(76) Inventor: Richard W. Gross, Chesterfield, MO (US)

Correspondence Address: Patrick W. Rasche;Armstrong Teasdale LLP, Ste 2600, One Metropolitan Square, St. Louis, MO, 63102-2740, US

(21) Appl. No.: 11/028,376

(22) Filed: Jan. 3, 2005

Related U.S. Application Data
(60) Provisional application No. 60/533,999, filed on Jan. 2, 2004.

(51) Int. Cl. A01K 67/027; A61K 38/46

(52) U.S. Cl. 424/94.6; 800/18

(57) ABSTRACT

A method for intentionally modulating by externally directing the amount of fat in a living mammal having adipocyte cells which comprises administering a compound thereto having reactive selectivity to the metabolic regulatory system of the cells of the animal to increase the fatty acid α oxidation of the lipid content in the differentiating adipocyte thereby reducing the amount of equivalents available for ATP synthesis.

This application claims the benefit of U.S. provisional patent application 60/533,999 filed Jan. 2, 2004 the contents of which are incorporated herein in their entirety by reference.

This research was supported by NIH Grant 5P01H57278-08. The government has certain rights in this invention.

### FIELD OF THE INVENTION

This discovery relates to a method of intentionally noninvasively therapeutically modulating fat in a living mammal. More particularly the invention relates to a useful screening method for identifying a compound useful to modulate fat in a living mammal such as a human. This invention also relates to a pharmacological target in screening potentially useful anti-obesity drugs and drugs for use in humans for other sequelae of the metabolic syndrome including at least one of atherosclerosis, diabetes and hypertension and to an animal model useful for such screening.

### BACKGROUND OF THE INVENTION

In many industrialized countries, the incidence of human obesity is unfortunately increasing. Moreover; human obesity is a common and costly nutritional problem in the United States. Obesity is characterized clinically by the accumulation of fat tissue (at times this is referred to as body fat content).

In humans, obesity is usually defined as a body fat content greater than 25% of the total weight for males, or greater than 30% of the total weight for females. Regardless of the cause of obesity, obesity is an ever present problem for Americans. But a fat content greater than about 18% for males and greater than about 22% for females can have untold consequences secondary to several mechanisms and disorders of metabolic function. For example, obesity can have a significant adverse impact on health care costs and provoke a higher risk of numerous illnesses, including heart attacks, strokes and diabetes.

Without being bound by theory, it is believed that obesity in humans results from an abnormal increase in white adipose tissue mass that occurs due to an increased number of adipocytes (hyperplasia) or from increased lipid mass accumulating in existing adipocytes. Obesity and the associated type two metabolic syndrome along with its clinical sequelae are among the major and the most rapidly increasing medical problems in America. However, to date, a lack of suitable adipocyte specific protein targets has unfortunately hampered progress in the development of effective therapeutic agents to combat the clinical sequelae of obesity.

Understanding the mechanisms underlying chemical energy storage and utilization in adipocytes is central to containing the epidemic of obesity currently afflicting industrialized nations⁽¹⁻⁴⁾. Adipocytes transport fatty acids across their plasma membranes where the carboxylic acid group is activated by ATP dependent thioesterification. The relative amount of thermal and chemical energy produced in cells is tightly regulated by modulating the coupling efficiency of fatty acid oxidation with ATP generation and heat production^{(5, 6)}. In general, in mitochondria fatty acid oxidation is usually tightly coupled to ATP synthesis while in peroxisomes fatty acid oxidation is loosely coupled generating heat at the expense of chemical energy. In mitochondria, this ratio is modulated by uncoupling proteins and intramitochondrial nonesterified fatty acids^{(5,6)} produced in part by iPLA₂β and iPLA₂γ.

Since de novo synthesis results in, and dietary sources contain, almost exclusively even chain length fatty acids, the overwhelming majority of lipids in mammalian cells are comprised of even numbered fatty acyl moieties. Some branched chain fatty acids (e.g. phytanic acid) must be α oxidized to produce the corresponding fatty acid with one less carbon atom to render the resulting branched chain backbone suitable for degradation by β oxidation. The first committed step in phytanic acid α oxidation is its 2-hydroxylation catalyzed by phytanoyl-CoA α hydroxylase followed by sequential oxidation and decarboxylation^{(12, 13)}. Prior studies have emphasized the exclusive role of this pathway in the metabolism of branched chain fatty acids since purified mammalian phytanoyl-CoA α hydroxylase failed to α oxidize unbranched long chain fatty acids⁽¹⁴⁾.

Accordingly, it was not believed that substantial α oxidation of unbranched long chain fatty acids occurred in mammalian cells. However, Mukherji et al recently demonstrated that purified peroxisomal phytanoyl-CoA α hydroxylase could α-hydroxylate unbranched long chain fatty acids in a test tube if sterol carrier protein 2 (SCP 2) was utilized to present the substrate⁽¹⁵⁾. However, definitive evidence for α-hydroxylation of unbranched long chain fatty acids in mammalian cells has not been forthcoming. Accordingly, the presence and amount of α oxidation of unbranched long chain fatty acid catabolism in eukaryotic cells is unknown. Moreover, all of the genes necessary for α oxidation of fatty acids are present in the human genome and are localized to the peroxisomal compartment by virtue of either an N terminal (type 2) or C terminal (type 1) peroxisomal localization signals^{(12,13, 16-19)}.

It is highly desired to be able to control fat in living animals. Enhanced clinical methodology and research tools and research methods are highly needed for identifying useful drugs to pharmacologically treat obesity and over-weightness. It is highly desired to discover technology based on the specific types of phospholipases present in the adipocyte or their mechanisms of regulation and determine their natural substrates and roles in anabolic lipid metabolism, catabolic lipid metabolism or both (e.g. triglyceride cycling).

Additionally, a screening method and research tool is needed to identify useful drugs which can be used to reduce the fat level of a living mammal and/or to maintain the fat level at a predetermined level. Also, a method is needed to effectively intentionally modulate the coupling of thermal and chemical energy in peroxisomes to decrease or increase the fat content of a living mammal.

### BRIEF DESCRIPTION OF THE DRAWINGS

FIG. 1. Alterations of phosphatidylserine molecular species during hormone-induced differentiation of 3T3-L1 cells.

FIG. 2. Alterations in phosphatidylinositol molecular species during hormone induced differentiation.

FIG. 3. Alterations of phosphatidylcholine molecular species from 3T3-L1 cells during hormone-induced differentiation of 3T3-L1 cells.

FIG. 4. Negative-ion electrospray ionization mass spectrometry and tandem mass spectrometry of ethanolamine glycerophospholipids from day 8 3T3-L1 cells by 2D approach described in U.S. Provisional Patent Application 10/606,601, filed Jun. 26, 2003, “Spectrometric Quantitation Of Triglyceride Molecular Species”.

FIG. 5. Positive-ion electrospray ionization mass spectrometry and tandem mass spectra of triglycerides from day 8 3T3-L1 cells.

FIG. 6. Determination of the double bond positions present in odd chain length moieties in TAG, PE and PC as illustrated by GC/EI/MS of their dimethyldisulfide adducts.

FIG. 7. Immunohistochemistry of SCD I in differentiated adipocytes.

FIG. 8. Fatty acid oxidation of exogeneously administered fatty acids in 3T3-L1 cells during differentiation.

FIG. 9. Messenger RNA levels of FACO and PAHX in 3T3-L1 cells during differentiation.

FIGS. 10-15 show sequences of phospholipases.

### DETAILED DESCRIPTION OF THE DRAWINGS

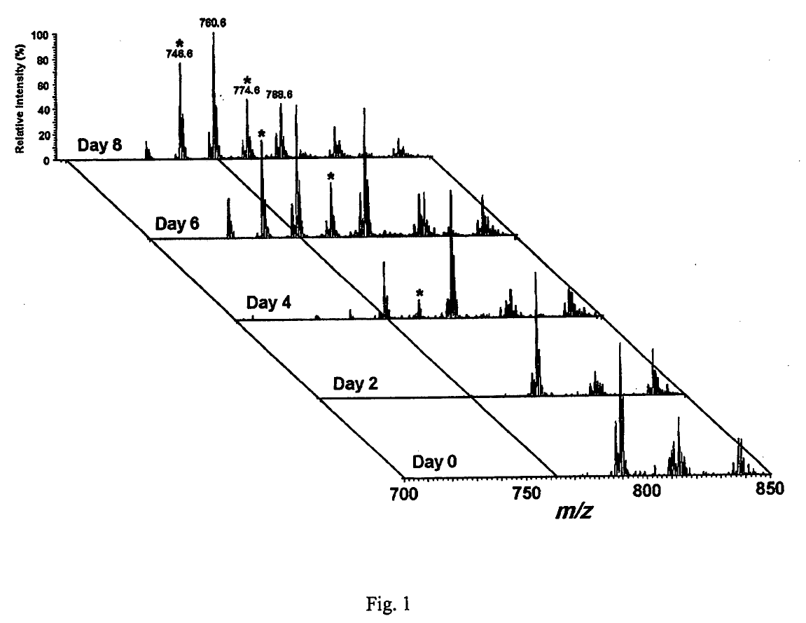

FIG. 1 shows alterations of phosphatidylserine molecular species during hormone-induced differentiation of 3T3-L1 cells. Lipids from 3T3-L1 cells at different stages of differentiation were extracted by a modified Bligh-Dyer technique utilizing 50 mM LiCl in the aqueous layer as described in “Test Procedures.” All samples were diluted in 1:1 methanol/chloroform prior to direct infusion into the ESI source using a syringe pump at a flow rate of 2 μL/min. Phosphatidylserine molecular species of 3T3-L1 cells at different stages of differentiation were fingerprinted by neutral loss scanning of 87.1 amu in the negative ion mode as described in “Test Procedures.” Mass spectra were normalized to the most intense peak in each individual spectrum. Asterisks indicate the molecular species containing odd chain length acyl moieties which are quantified in Table II.

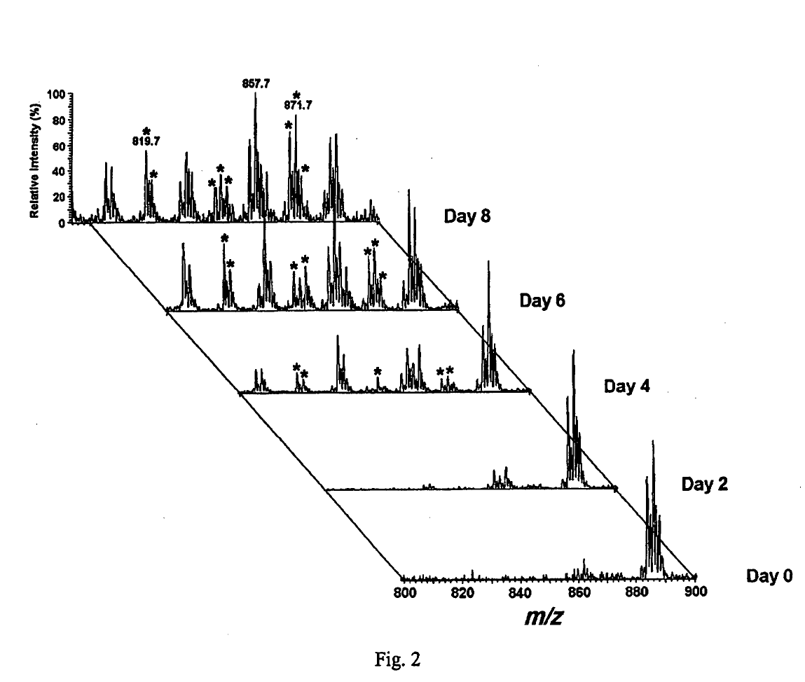

FIG. 2 shows alterations of phosphatidylinositol molecular species during hormone-induced differentiation of 3T3-L1 cells. Lipids from 3T3-L1 cells at different stages of differentiation were extracted by a modified Bligh-Dyer technique utilizing 50 mM LiCl in the aqueous layer as described in “Test Procedures.” All samples were diluted in 1:1 methanol/chloroform prior to direct infusion into the ESI source using a syringe pump at a flow rate of 2 μL/min. Phosphatidylinositol molecular species were fingerprinted by precursor ion scanning of m/z 241.2 in the negative ion mode as described in “Test Procedures.” Mass spectra were normalized to the most intense peak in each individual spectrum. Asterisks indicate the molecular species containing odd chain length acyl moieties which are quantified in Table I.

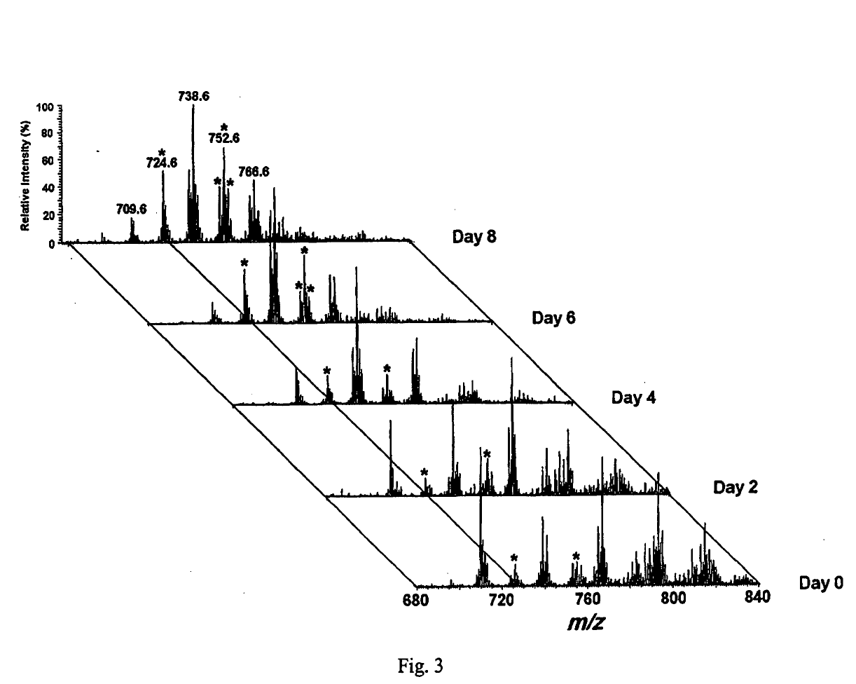

FIG. 3 shows alterations of phosphatidylcholine molecular species from 3T3-L1 cells during hormone-induced differentiation of 3T3-L1 cells. Lipids from 3T3-L1 cells at different stages of differentiation were extracted by a modified Bligh-Dyer technique utilizing 50 mM LiCl in the aqueous layer as described in “Test Procedures.” All samples were diluted in 1:1 methanol/chloroform and LiOH (50 nmol/mg protein) was added just prior to direct infusion into the ESI source using a syringe pump at a flow rate of 2 μL/min. Phosphatidylcholine molecular species were visualized by neutral loss scanning of 183.2 amu in the positive ion mode. All neutral loss mass spectra were displayed after normalization to the most intense peak in the individual spectrum. Asterisks indicate the molecular species containing odd chain length acyl moieties which are quantified in Table III.

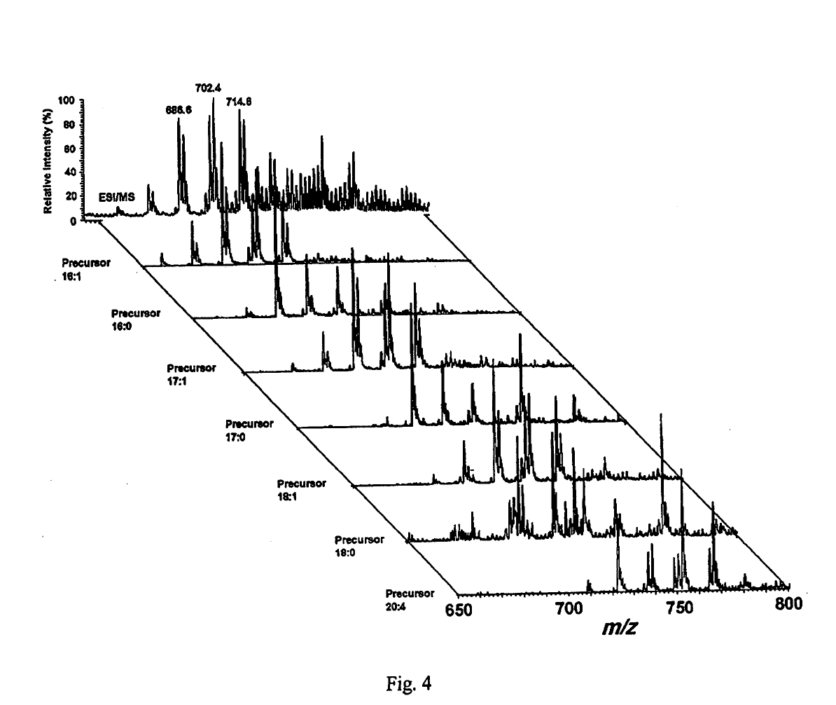

FIG. 4 shows negative-ion electrospray ionization mass spectrometry and tandem mass spectrometry of ethanolamine glycerophospholipids from day 8 3T3-L1 cells. Lipids from day 8 3T3-L1 cells were extracted by a modified Bligh-Dyer technique utilizing 50 mM LiCl in the aqueous layer as described in “Test Procedures.” All samples were diluted in 1:1 methanol/chloroform with addition of LiOH (50 nmol/mg protein) just prior to their direct infusion into the ESI source using a syringe pump at a flow rate of 2 μL/min. PE molecular species were identified by precursor-ion scanning of m/z 253.2 (16:1), 255.2 (16:0), 267.2 (17:1), 269.2 (17:0), 281.2 (18:1), and 303.2 (20:4) in the negative-ion mode. All precursor ion scanning mass spectra were displayed after normalization to the most intense peak in each individual spectrum.

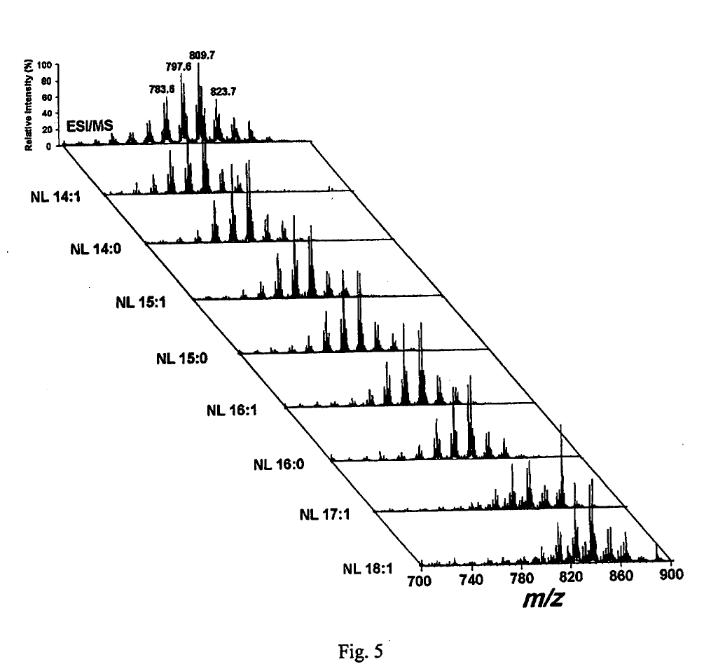

FIG. 5 shows positive-ion electrospray ionization mass spectrometry and tandem mass spectra of triglycerides from day 8 3T3-L1 cells. Lipids from day 8 3T3-L1 cells were extracted by a modified Bligh-Dyer technique utilizing 50 mM LiCl in the aqueous layer as described in “Test Procedures.” All samples were diluted in 1:1 methanol/chloroform with addition of LiOH (50 nmol/mg protein) just prior to direct infusion into the ESI source using a syringe pump at a flow rate of 2 μL/min. Positive-ion ESI mass spectra of triglycerides in lipid extracts was acquired as described in “Test Procedures.” Positive-ion ESI tandem mass spectra by neutral loss scanning of 226.2 (14:1), 228.2 (14:0), 240.2 (15:1), 242.2 (15:0), 254.2 (16:1), 256.2 (16:0), 268.2 (17:1) and 282.2 (18:1) amu were acquired as previously described in “Test Procedures.” All neutral loss scanning mass spectra were displayed after normalization to the most intense peak in the individual spectrum.

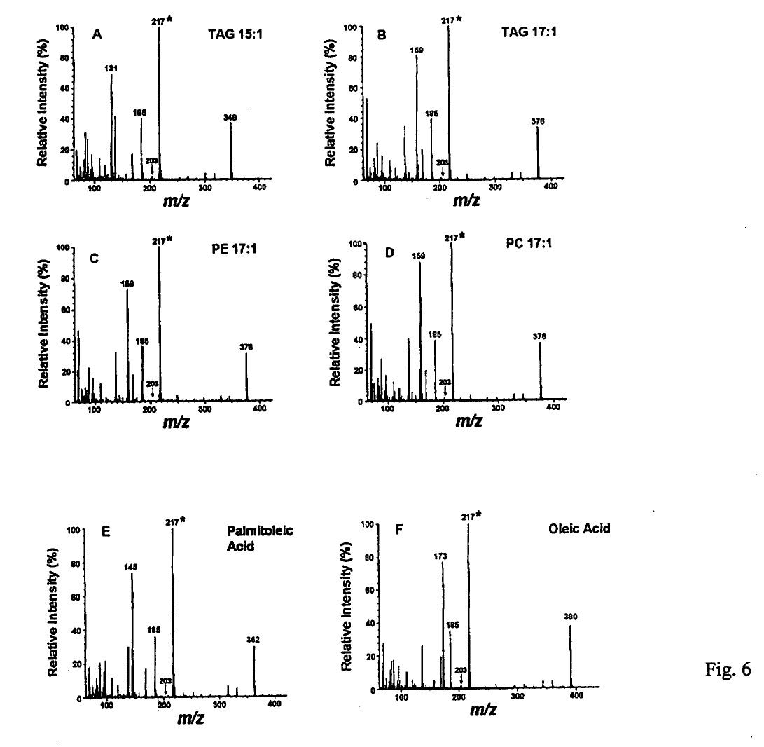

FIG. 6 shows determination of the double bond positions present in odd chain length moieties in TAG, PE and PC as illustrated by GC/EI/MS of their dimethyldisulfide adducts. Lipids from day 8 3T3-L1 cells were extracted by the method of Bligh-Dyer utilizing 50 mM LiCl in aqueous phase. Lipid classes were resolved from the chloroform extract utilizing an Ultrasphere silica HPLC column by gradient elution at a flow rate of 2 mL/min as described in “Test Procedures.” The acyl moieties in TAG, PE and PC were liberated as their methyl esters by acid methanolysis. The resulting monounsaturated fatty acyl methyl esters from each lipid class were converted to their dimethyldisulfide (DMDS) adducts by incubation with dimethyldisulfide and I₂. The resulting DMDS adducts were extracted with hexane and analyzed by GC/MS as described in “Test Procedures.” A, EI/MS spectra of the C15:1 methyl ester DMDS adduct from TAG. B, EI/MS spectra of the C17:1 methyl ester DMDS adduct from TAG. C, EI/MS spectra of the C17:1 methyl ester DMDS adduct from PE. D, EI/MS spectra of the C17:1 methyl ester DMDS adduct from PC. E, EI/MS spectra of the palmitoleic acid methyl ester DMDS adduct from lipid mixture. F, EI/MS spectra of the oleic acid methyl ester DMDS adduct from lipid mixture. In all the spectra, asterisks indicate m/z 217, which results from Δ9 fatty methyl ester DMDS adducts. The positions of m/z 203, which would result from Δ8 fatty methyl ester DMDS adducts but not present in significant amounts, are indicated by arrows.

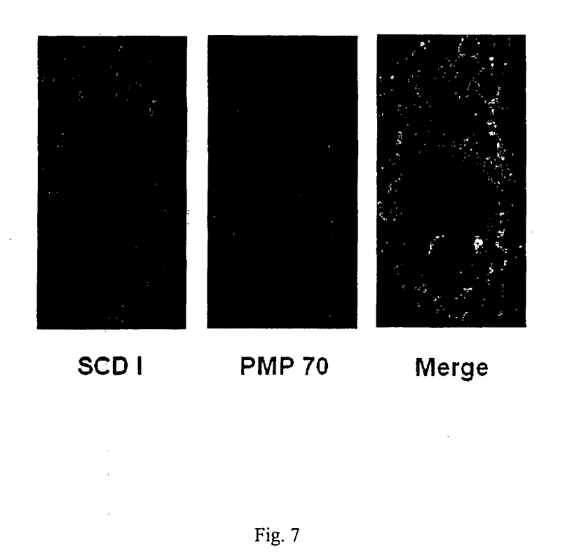

FIG. 7 shows immunohistochemistry of SCD I in differentiated adipocytes. Six days after differentiation 3T3-L1 adipocytes were replated at 30 to 50% of their original density. Adipocytes were fixed for 10 minutes with 2% formaldehyde in phosphate-buffered saline (PBS). The coverslips were rinsed in PBS and then incubated for 1 hour in both PMP70 antiserum (diluted 1:500 in microscopy buffer) and anti-stearoyl-CoA dehydrogenase (1 μg/mL). The coverslips were rinsed in PBS and then incubated for 30 minutes with 594 donkey anti-rabbit IgG and 488 Donkey anti-goat IgG both diluted 1:1000 in microscopy buffer. SCD I (left panel); PMP 70 (middle panel); Merged (right panel)

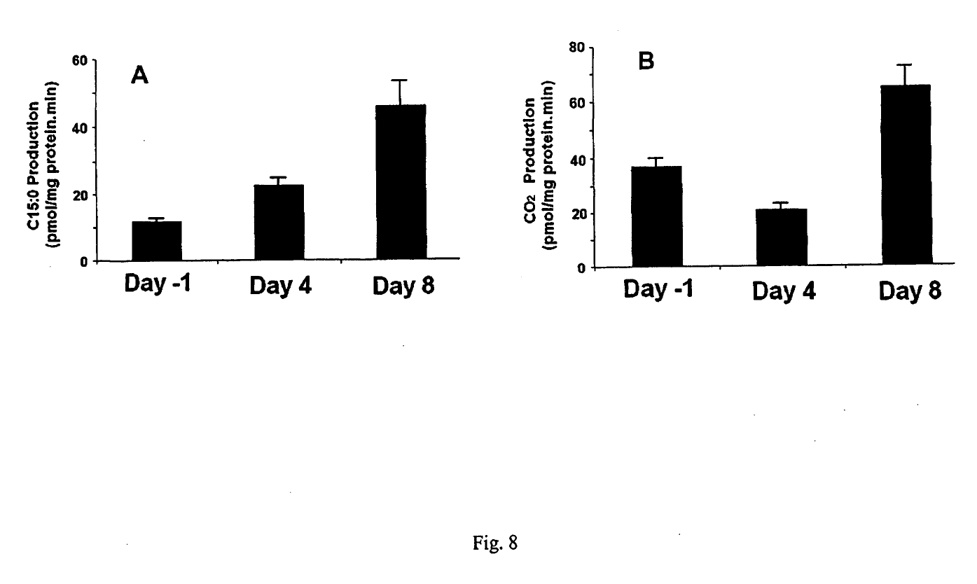

FIG. 8 shows fatty acid oxidation of exogeneously administered fatty acids in 3T3-L1 cells during differentiation. A, 3T3-L1 cells at selected times during differentiation were radiolabeled by incubation with 5 μCi of [9,10-³H]-palmitic acid by ethanolic injection into the incubation media. After 0.5, 1, 2, and 5 hours, the media was removed, the cells were washed with PBS, scraped into 1.5 mL deionized water, and total lipids were extracted according to the method of Bligh-Dyer with 1% acetic acid in the aqueous layer. The initial rates of radiolabeled C16:0 into C15:0 fatty acids were quantified as described under “Test Procedures.” Unlabeled non-esterified fatty acids were purified and their concentrations were determined as described in “Test Procedures.” The α oxidation rate was approximated by estimating the initial flux of C16:0 to C15:0 mass as described under “Test Procedures.” B, 3T3-L1 cells were cultured in T-25 flasks to confluence and induced to differentiate. At selected differentiation stages, [1-¹⁴C]-palmitate was added to a final concentration of 200 nCi/mL. The flasks were sealed and fitted with a center well comprised of Whatman No. 1 filter paper. At selected time points, the ¹⁴CO₂ liberated from the medium by acidification with 2 mL of 6 M of HCl was collected on the filter paper previously alkalinized with 250 μL of 2 M NaOH. Released radiolabeled ¹⁴CO₂ was quantified by liquid scintillation counting. The fatty acid oxidation rate was approximated by calculating the initial rate of production of ¹⁴CO₂ as described under “Test Procedures.”

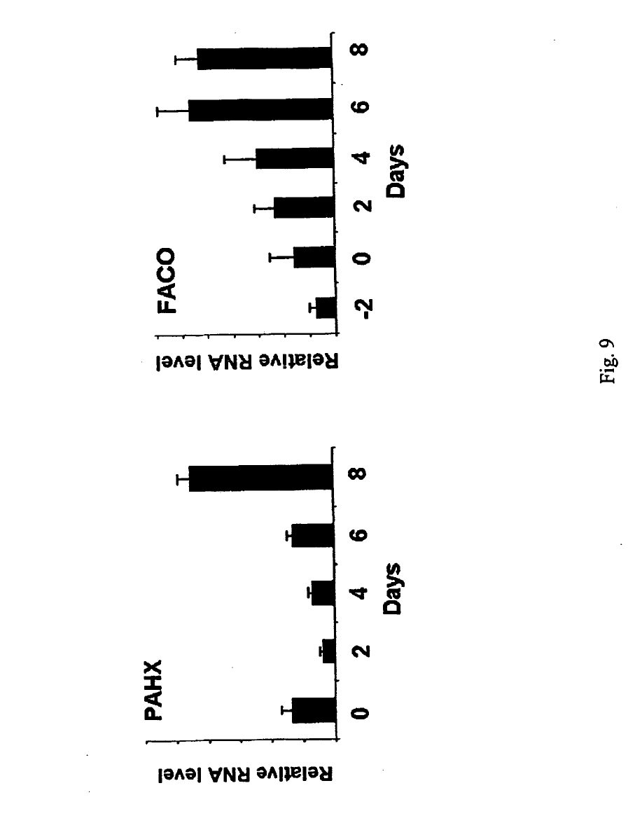

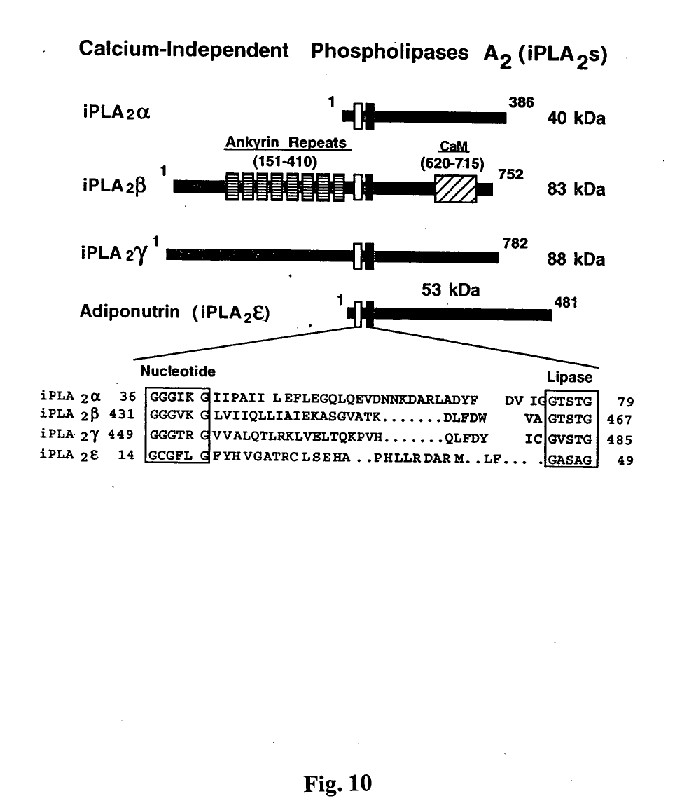

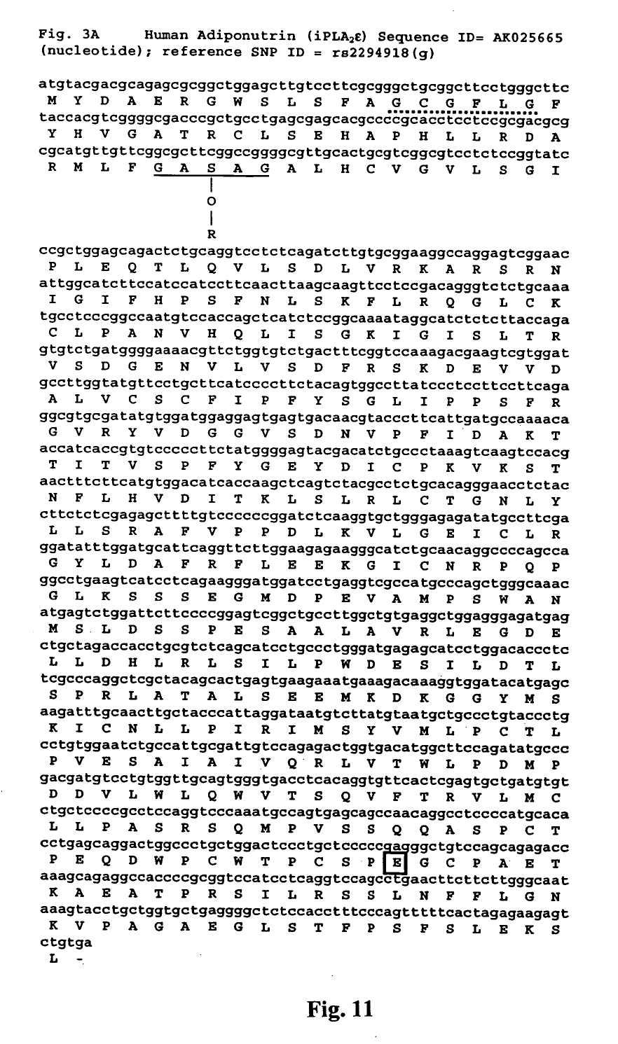

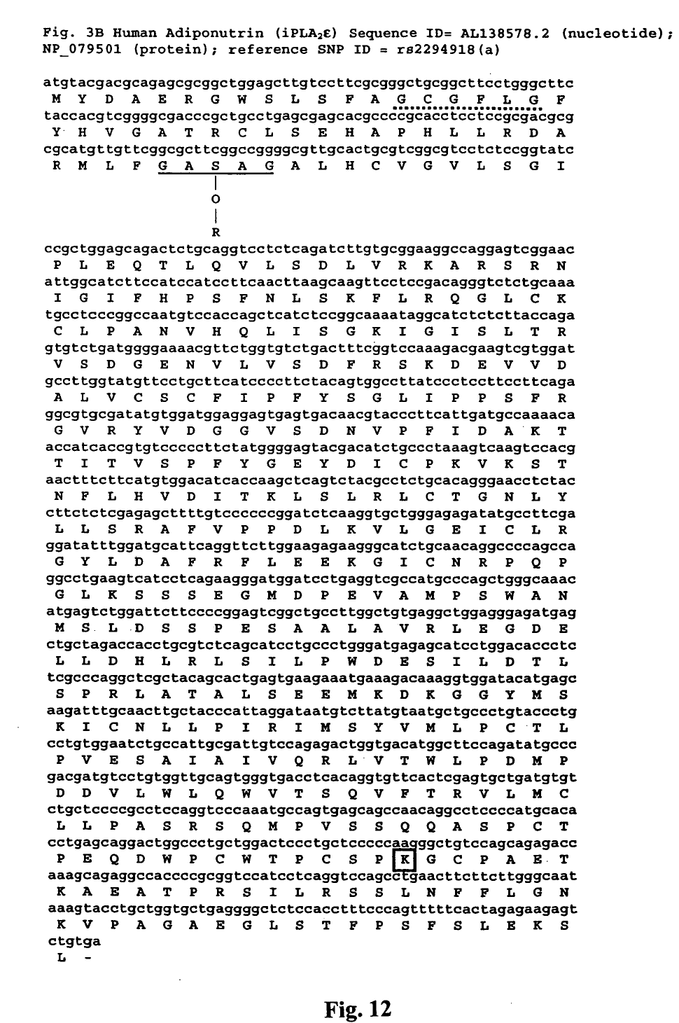

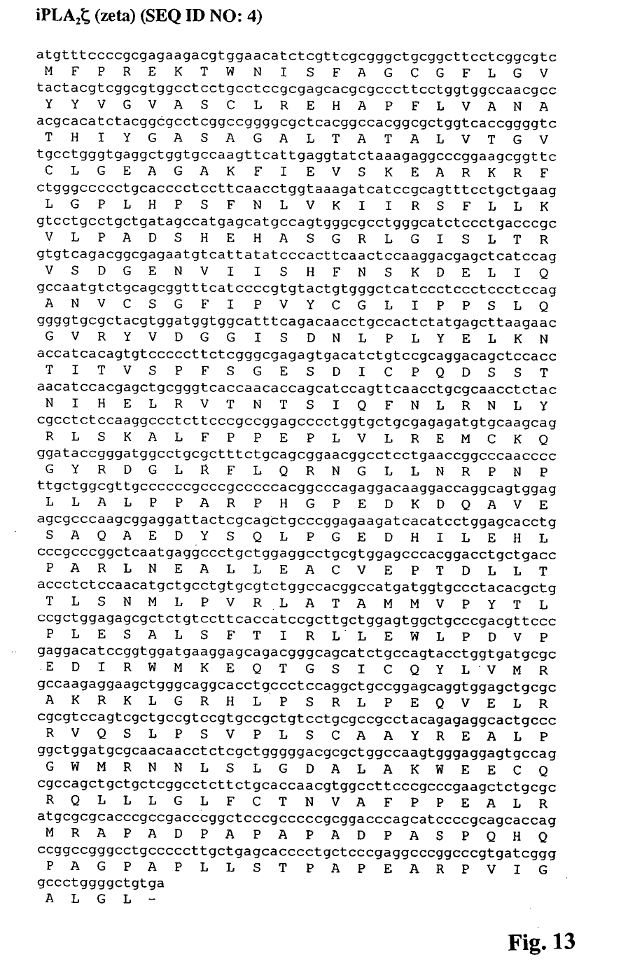

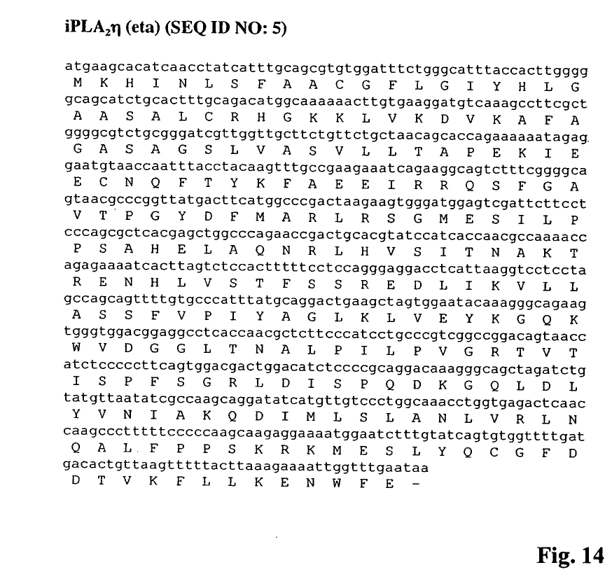

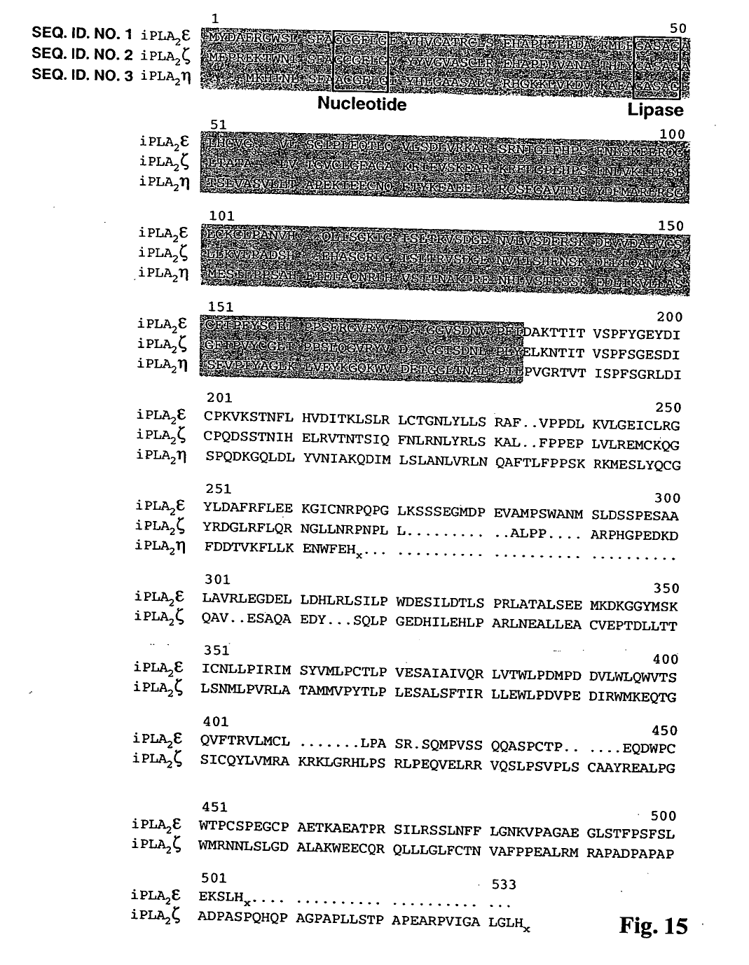

FIG. 9 shows messenger RNA levels of FACO and PAHX in 3T3-L1 cells during differentiation. 3T3-L1 cells were cultured and induced to differentiation as described in “Test Procedures.” At indicated differentiation stages, total RNA was prepared as described in “Test Procedures.” Quantitative PCR was performed with TaqMan® PCR reagent kits in the ABI PRISM 7700 detection system utilizing GAPDH as the internal standard. A representative result of three independent tests is shown.

### BRIEF DESCRIPTION OF THE INVENTION

The inventor discovered a method for intentionally noninvasively modulating the amount of fat in a living mammal having adipocyte cells which comprises effectively administering a compound thereto (such as administration to a mammal), the compound having reactive selectivity to the metabolic regulatory system of the cells of the animal to increase the fatty acid α oxidation of the adipocyte thereby reducing the amount of energy available for ATP synthesis and increasing heat production for each molecule of FA taken up by the fat cell thereby reducing the amount of fat in the animal. In an aspect, the amount of fat is decreased. In an aspect, the amount of fat is increased. In an aspect, the amount of fat is decreased to lower the risk or to treat diabetes by decreasing fat content. In an aspect, more or less of a compound is administered to the animal living model to modulate the amount of fat. In a further aspect, the amount of compound effectively administered is that amount (therapeutic) which correspondingly produces the desired responsive increase or decrease in fat content of the mammal. In an aspect the amount of fat is reduced.

To identify the principle metabolic pathways regulating fatty acid flux in adipocytes, alterations in the lipidome of 3T3-L1 cells (mouse, Swiss-albino, embryo) during their hormone-induced differentiation into adipocytes were determined by electrospray ionization mass spectrometry (ESI/MS/MS). This model of adipocyte differentiation recapitulates the biochemical, molecular biologic and ultrastructural characteristics of differentiated adipocytes in mammals including dramatic increases in intracellular triglyceride (TAG) content, augmented transcription and translation of adipocyte specific genes and the morphologic appearance of adipocytes⁽⁷⁻¹⁰⁾. Moreover, compelling studies have demonstrated that this cell lineage can differentiate into adipocyte tissue that cannot be distinguished from existing adipocytes when grafted into athymic mice⁽¹¹⁾. A small fractional difference in caloric expenditure over each cell's lifetime have substantial effects on the total mass of accumulated lipids, a role for α oxidation as a modulator of adipocyte lipid mass seems possible.

In an aspect, an animal model has been discovered and is presented herein which is useful for identifying a compound(s) which modulates the amount of fat in a living mammal and which presents a target for identifying compounds mediating fatty acid α oxidation a selection of tissue having fatty acid α oxidation capability in the peroxisomal compartment of cells of that tissue. In an aspect, the compound is externally effectively administered to a living animal model in a beneficial therapeutic amount.

In an aspect, the inventive method comprises directing or modulating a biochemical pathway of a mammal by administering a compound effectively to the mammal so as to direct or modulate, wherein the pathway comprises a fatty acid α oxidation in a cell peroxisomal compartment. In a further aspect, an animal model is provided for this direction and/or modulation.

In an aspect, a method for identifying a compound which modulates the amount of fat in a living mammal which presents itself as target for a compound mediating fatty acid an α oxidation a selection of tissue having fatty acid α oxidation capability in the peroxisomal compartment of cells of that tissue comprises administering a candidate compound to that living tissue and measuring α oxidation rates or surrogates thereof, determining that the compound is a modulator if α oxidation is increased leading to heat production and weight loss. In an aspect, the mammal has pre-adipocyte cells. In an aspect, the administration of a compound is an intentional non-invasive intervention in the biosystem bioprocesses of the living model to increase, decrease or hold fat content.

In an aspect, an isolated, characterized purified target is presented for intentional externally directed modulated bio interaction with an externally administered complex which categorizes α oxidation, comprising tissue having fatty acid α oxidation capability in the peroxisomal compartment of a cell wherein that interaction results in a modulation of fat content. In an aspect, the cell is an isolated cell. In an aspect, the cell(s) is a functional model replicate of a living mammal. In an aspect the cell(s) comprises a living mammal. In an aspect the intervention is prescripted.

In an aspect, an isolated, characterized and purified target for regulating fat is presented in a living mammal which comprises methods for altering the relative flux of fatty acids to the peroxisomal compartment leading to heat or to the mitochondrial compartment in proximity to catabolic synthetic pathways.

In an aspect, fatty acid is intentionally released from parent lipid (non polar or polar lipid) to the peroxisome in the biosystem of a living mammal for alpha oxidation and reduction of fat content by the administration of an adipocyte phospholipase to the molecular regulatory biosystem of a living mammal, the phospholipase including but not limited to phospholipase gamma, phospholipase epsilon, phospholipase zeta or phospholipase eta and provoking the subsequent alpha oxidation of the released fatty acid thereby reducing the lipid content in the animal and reducing overweightness of the living mammal.

### DETAILED DESCRIPTION OF THE INVENTION

This discovery relates to a method of intentionally therapeutically modulating fat in a living mammal. More particularly the invention relates to a useful screening method for identifying compounds useful to modulate fat in a living mammal such as a nonhuman animal. This invention also relates to a pharmacological target in screening potentially useful anti-obesity drugs and drugs for other sequelae of the metabolic syndrome including at least one of atherosclerosis, diabetes and hypertension and to an animal model useful for such screening.

A paper by Richard W. Gross, Xiong Su and Xianlin Han references “Proceedings of the 50^{th} ASMS Conference on Mass Spectrometry and Allied Topics, Orlando, Fla., Jun. 2-6, 2002” presented information on “Accumulation of Odd Chain Length Acyl Moieties in 3T3-L1 Cells during Insulin-Induced Differentiation to Fat Cells; Identification of α-Oxidation in Mammalian Cells”. This paper is incorporated herein in its entirety by reference.

The inventor discovered that α oxidation in a living mammal using unbranched fatty cells produces CO₂ at the expense of acetyl CoA production in an uncoupled heat producing reaction and thus does not participate in the efficient conversion of fatty acid into chemical fuel leading to lipid storage. This discovery utilizes fatty acid α oxidation as an important metabolic determinant of the lipid content in mammalian cells.

The inventor has discovered a method to specifically, intentionally and deliberately cause activated fatty acids to be utilized in an increased manner and amount for inefficient oxidative catabolism resulting in increased production of heat and decreased chemical energy (e.g. ATP).

Since α oxidation utilizes O₂ as a direct electron acceptor, this reaction does not produce any reducing equivalents which can be utilized for ATP synthesis. Thus, α oxidation in the peroxisomal compartment is constitutively uncoupled and serves as a de facto mechanism to shed the Gibbs free energy inherit in the C—C bond of fatty acids (i.e. thermogenesis). Moreover, α oxidation produces CO₂ at the expense of acetyl CoA production and thus does not participate in the efficient conversion of fatty acid into chemical fuel for cellular utilization. Thus, these results indicate a prominent role for fatty acid α oxidation as an important metabolic determinant of the lipid content in the differentiating adipocyte. Collectively, these results underscore the role of fatty acid α oxidation in the peroxisomal compartment as a proximal mechanism which modulates fatty acid metabolic fate and energy storage in the differentiating adipocyte.

The inventor discovered that alpha oxidation promotes free radical toxicity from the obligatory production of H₂O₂ which is also known to be deleterious to the human condition in excess. It is the precise balance of α and β oxidation as well as the peroxisomal and mitochondrial pathways which are critical for human health.

Activated fatty acids can subsequently be incorporated into lipid storage pools (e.g., TAG) or alternatively be utilized as substrates for oxidative catabolism resulting in production of heat and chemical energy (e.g., ATP). The inventor has discovered a method for intentionally modulating the coupling of thermal and chemical energy in peroxisomes whereby fatty acids are intentionally utilized more extensively in inefficient oxidative catabolism. In an aspect, the amount of such inefficient catabolism is intentionally increased.

During the course of his studies on alterations in lipid metabolism in differentiating adipocytes, the inventor identified unanticipated time-dependent accumulation of odd chain length fatty acyl moieties in multiple phospholipid classes and triglycerides. To explore the biochemical mechanisms underlying this phenomenon, a detailed lipid class and molecular species analysis utilizing electrospray ionization mass spectrometry was performed in conjunction with kinetic radiolabeling tests to gain insight into the biochemical mechanisms underlying the changes in lipid metabolism in the 3T3-L1 differentiating adipocyte. Herein, the inventor reports the identification of robust α oxidation of unbranched long chain fatty acids in a mammalian cell line and the obligatory ordered sequential α oxidation of fatty acids prior to their Δ9 desaturation. In essence, these results demonstrate that Δ9 desaturated fatty acids do not serve as substrates for α oxidation. Collectively, the results demonstrate the importance of proximal peroxisomal fatty acid processing as an important determinant of the metabolic fate of fatty acids in the adipocyte.

Further, the inventor discovered an useful medical treatment for effectively combating obesity and over-weightness in humans.

The inventor discovered a screening method useful to identify compounds which modulate peroxisomal fatty acid metabolism in general and peroxisomal fatty acid α oxidation specifically to utilize to screen libraries for drugs which can be used successfully to hold weight in a living mammal or if desired to induce weight loss through increasing peroxisomal processing of fatty acids and/or their metabolism by α oxidative pathways.

Herein, the inventor provides his method to exploit the power of global lipidomics to identify the critical role of peroxisomal processing of fatty acids in adipocyte lipid storage and metabolism. Remarkably, 3T3-L1 differentiating adipocytes rapidly acquired the ability to α oxidize unbranched fatty acids as manifest by the accumulation of odd chain-length unbranched fatty acids in all major lipid classes. Moreover, in differentiating adipocytes, unsaturated odd chain-length fatty acids in triglyceride molecular species contained exclusively Δ9 olefinic linkages. Unsaturated fatty acids (e.g. oleic and palmitoleic acids) were not subject to α oxidation resulting in the absence of Δ8 unsaturated odd chain length fatty acids. This highly selective substrate utilization resulted in the obligatory sequential ordering of α oxidation prior to Δ9 desaturation. Based on these results, a putative type 2 peroxisomal localization sequence was identified at the N terminus of mouse stearoyl CoA desaturase I (SCD I) comprised of ³⁰KVKTVPLHL³⁸. Kinetic analysis demonstrated that the rate of α oxidation of exogenously administered [9,10-³H] palmitic acid increased 4-fold during differentiation. Similarly, quantitative PCR demonstrated a 4-fold increase in phytanoyl-CoA αhydroxylase (PAHX) and fatty acyl-CoA oxidase (FACO) mRNA levels during differentiation. Collectively, these results underscore the role of peroxisomal fatty acid processing as an important determinant of the metabolic fate of fatty acids in the differentiating adipocyte.

The inventor discovered a fundamental biochemical process in mammalian fat cells which is a screening method to identify a compound(s) which modulate peroxisomal fatty acid metabolism and/or α oxidation to identify pharmaceutical agents to increase weight in a living mammal by inhibiting α oxidation thereby increasing fat stores and thus decreasing serum lipids, decreasing insulin requirements and attenuating the effects of atherosclerosis.

As used herein, the term “compound” includes cell(s), compounds, ions/anions, cations and salts.

As used herein, the term “adipocyte” indicates any living cell which stores fat.

As used herein, the term “tissue” includes tissue, cells and collections of a multiplicity of homogenous or nearly homogenous cell lines or a sample thereof or a representative sample thereof. In an aspect the tissue is a living mammalian tissue such as in a tissue culture or living mammal or in a living transgenic mouse.

As used herein, the term “peptide” is any of a group of compounds comprising two or more amino acids linked by chemical bonding between their respective carboxyl and amino groups. The term “peptide” includes peptides and proteins that are of sufficient length and composition to affect a biological response, e.g. antibody production or cytokine activity whether or not the peptide is a hapten. The term “peptide” includes modified amino acids, such modifications including, but not limited to, phosphorylation, glycosylation, acylation prenylation, lipidization and methylation.

As used herein, the term “polypeptide” is any of a group of natural or synthetic polymers made up of amino acids chemically linked together such as peptides linked together. The term “polypeptide” includes peptide, translated nucleic acid and fragments thereof.

As used herein, the term “polynucleotide” includes nucleotide sequences and partial sequences, DNA, cDNA, RNA variant isoforms, splice variants, allelic variants and fragments thereof.

As used herein, the terms “protein”, “polypeptide” and “peptide” are used interchangeably herein when referring to a translated nucleic acid (e.g. a gene product). The term “polypeptide” includes proteins.

As used herein, the term “isolated polypeptide” includes a polypeptide essentially and substantially free from contaminating cellular components.

As used herein, the term “isolated protein” includes a protein that is essentially free from contamination cellular components normally associated with the protein in nature.

As used herein, the term “nucleic acid” refers to oligonucleotides or polynucleotides such as deoxyribonucleic acid (DNA) and ribonucleic acid (RNA) as well as analogs of either RNA or DNA, for example made from nucleotide analogs any of which are in single or double stranded form.

As used herein, the term “patient” and subject” are synonymous and are used interchangeably herein.

As used herein, the term “expression” includes the biosynthesis of a product as an expression product from a gene such as the transcription of a structural gene into mRNA and the translation of mRNA into at least one peptide or at least one polypeptide.

As used herein, the term “mammal” includes living animals including humans and non-human mammals such as murine, porcine, canine and feline.

As used herein, the term “sample” means a viable sample of biological tissue or fluid and is not limited to heart tissue. Biological samples may include representative sections of tissues.

As used herein, the term “antisense” means a strand of RNA whose sequence of bases is complementary to messenger RNA.

As used herein, the term “siRNA” means short interfering RNA.

As used herein, “ATP” means adenosine triphosphate.

As used herein, “ADP” means adenosine diphosphate.

The phrase “a sequence encoding a gene product” refers to a nucleic acid that contains sequence information, e.g., for a structural RNA such as rRNA, a tRNA, the primary amino acid sequence of a specific protein or peptide, a binding site for a transacting regulatory agent, an antisense RNA or a ribozyme. This phrase specifically encompasses degenerate codons (i.e., different codons which encode a single amino acid) of the native sequence or sequences which may be introduced to conform with codon preference in a specific host cell.

By “host cell” is meant a cell which contains an expression vector and supports the replication or expression of the expression vector. Host cells may be prokaryotic cells such as E. coli, or eukaryotic cells such as yeast, insect, amphibian, e.g. Xenopus, or mammalian cells such as HEK293, CHO, HeLa and the like.

As used herein a “therapeutic amount” is an amount of a moiety such as a drug or compound which produces a desired or detectable therapeutic effect on or in a mammal administered with the moiety.

The term “recombinant” when used with reference to a cell, or protein, nucleic acid, or vector, includes reference to a cell, protein, or nucleic acid, or vector, that has been modified by the introduction of a heterologous nucleic acid, the alteration of a native nucleic acid to a form not native to that cell, or that the cell is derived from a cell so modified. Thus, for example, recombinant cells express genes and proteins that are not found within the native (non-recombinant) forms of the cell or express native genes that are otherwise abnormally expressed, under expressed or not expressed at all.

In an aspect, administering a compound that regulates the expression, mass or activity of endogenous fat cell phospholipases/lipases (e.g., iPLA2beta, iPLA2gamma, iPLA2epsilon, iPLA2zeta, iPLA2eta) and other lipases that provides substrate for alpha oxidation of FA to facilitate the removal of fat from the adipocyte.

In an aspect administering a compound that regulates the expression, mass or activity of endogenous mammalian cell phospholipases or lipases (e.g., iPLA2beta, iPLA2gamma, iPLA2epsilon, iPLA2zeta, iPLA2eta) to facilitate the removal of fat or toxic fatty acyl moieties from the targeted cell (e.g., muscle cell, pancreatic beta cell, or liver cell) by alpha oxidation.

In a further aspect, transfectamine is obtained from InVitrogen Corporation, 1600 Faraday Avenue, P.O. Box 6482, Carlsbad, Calif. 92008 and/or Calgene Inc., 1920 Fifth Street, Davis, Calif. 95616 and a mixture is prepared in a tissue culture. This tissue culture is taken in tumor cells which “eat” the admixture comprising siRNA. Without being bound by theory it is believed that siRNA is an effective toxic agent against a gene comprising a polynucleotide. A useful web site, http://www.white head.mit.edu/nap/features/nap_feature_sirna.html provides technical information from Whitehead Biocomputing group which provides useful tools for identifying siRNA using a sequence of a polynucleotide. This information is incorporated herein in its entirety by reference.

The construction of a suitable vector can be achieved by any of the methods well-known in the art for the insertion of exogenous DNA into a vector, see Sambrook et al., 1989, Molecular Cloning, A Laboratory Manual, Cold Spring Harbor Press, N.Y.; Rosenberg, et al., Science 242:1575-1578 (1988); Wolff et al., Proc. Natl. Acad. Sci. USA 86:9011-9014 (1989). For Systemic administration with cationic liposomes, and administration in situ with viral vectors, see Caplen et al., Nature Med., 1:39-46 (1995); Zhu et al., Science, 261:209-211 (1993); Berkner et al., Biotechniques, 6:616-629 (1988); Trapnell et al., Advanced Drug Delivery Rev., 12:185-199; Hodgson et al., BioTechnology 13:222 (1995).

In an aspect iPLA₂ε can function as a signaling enzyme to control eicosanoid synergies and other lipids and messengers of signal transduction. In an aspect a method of attenuating eicosanoid synergies, lipids and messengers of signal transduction by increasing or decreasing the amount of iPLA₂ε present in a living biosystem such as a living mammal.

In an aspect, fatty acid is released from parent lipid (non polar or polar lipid) to the peroxisome for alpha oxidation and reduction of fat content by effective administration of an adipocyte phospholipase to the molecular regulatory biosystem of the living mammal, the phospholipase including but not limited to phospholipase gamma, phospholipase epsilon iPLA₂Epsilon, phospholipase zeta or phospholipase eta and provoking the subsequent alpha oxidation of the released fatty acid thereby reducing the lipid content in an animal and reducing overweightness of the living mammal. Through the action of these lipases which hydrolyze polar and non-polar lipids fatty acids are liberated which in the presence of active alpha oxidation can be metabolized with the production of heat thereby removing lipid content from the fat cell. In an aspect one can then use molecular biologic or pharmacologic methods to increase the activity of these phospholipases which in the presence of alpha oxidation would remove the lipid burden. Alternatively, both alpha oxidation of released fatty acids and increased lipase activity could be coordinately activated by either molecular biologic or pharmacologic means to synergistically increase the removal of lipids from a fat cell or another type of cell containing unwanted and potentially toxic fat deposits and fatty acids.

The above phospholipases are described in: (a) The Journal of Biological Chemistry, 2004 by The American Society of Biochemistry and Molecular Biology, Inc. Identification, Cloning, Expression, and Purification of Three Novel Human Calcium-independent Phospholipase A₂ Family Members Possessing Triacylglycerol Lipase and Acylglycerol Transacylase Activities*, Published, JBC Papers in Press, Sep. 10, 2004, DOI 10.1074/jbc.M407841200. Christopher M. Jenkins, David J. Mancuso, Wei Yan, Harold F. Sims, Beverly Gibson, and Richard W. Gross‡. This paper is available on-line at http://wwwjbc.org. This paper is incorporated herein in its entirety by reference.

(b) Mancuso, D. J., Jenkins, C. M., Sims, H. F., Cohen, J. M., Yang, J., and Gross, R. W.:Complex transcriptional and translational regulation of iPLA2g resulting in multiple gene products containing dual competing sites for mitochondrial or peroxisomal localization. Eur. J. Biochem. 271:4709-4724, 2004. This paper is incorporated herein in its entirety by reference.

(c) Mancuso, D. J., Jenkins, C. M., and Gross, R. W.: The genomic organization, complete mRNA sequence, cloning and expression of a novel human intracellular membrane-associated calcium-independent phospholipase A2. J. Biol. Chem. 2000;275:9937-9945. This paper is incorporated herein in its entirety by reference.

Phospholipase epsilon is described in U.S. Provisional Patent Application No. 60/528,951 filed Dec. 11, 2003 and in U.S. Nonprovisional patent application “Human iPLA₂ε” filed Dec. 13, 2004 under Certificate of Mailing No. EV459190420US having Attorney Docket No. 15060-51, which is incorporated herein in its entirety by reference.

Phospholipase eta and phospholipase zeta are also described in U.S. Provisional Patent Application 60/586,913 filed Jul. 9, 2004 which is incorporated herein in its entirety by reference.

In an aspect, the specific mammalian dose of an inhibitor compound or chemical is an effective amount according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. The amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

Compounds used will likely be in a pharmacologically effective amount pharmaceutical grade preferred, (including immunologically reactive fragments) are administered to a subject such as to a living patient using standard effective administration techniques, preferably peripherally (i.e. not by administration into the central nervous system) by intravenous, intraperitoneal, subcutaneous, pulmonary, transdermal, intramuscular, intranasal, buccal, sublingual, pill or suppository administration.

The compositions for effective administration to a living mannal are designed to be appropriate for the selected mode of administration, and pharmaceutically acceptable excipients such as compatible dispersing agents, buffers, surfactants, preservatives, solubilizing agents, isotonicity agents, stabilizing agents and the like are used as appropriate. Remington's Pharmaceutical Sciences, Mack Publishing Co., Easton Pa., USA 16Ed. ISBN: 0-912734-04-3, latest edition, incorporated herein by reference in its entirety, provides a compendium of formulation techniques as are generally known to practitioners.

The following examples are illustrative and are not meant to be limiting of this discovery in any way.

Test Procedures

3T3-L1 cells were obtained from ATCC (Manassas, Va.). DMEM and calf serum were purchased from Life Technologies, Inc. (Rockville, Md.) while fetal bovine serum was obtained from BioWhittaker, Inc. (Walkersville, Md.). Reagents for reverse transcription and quantitative polymerase chain reaction (PCR) were purchased from Applied Biosystems (Foster City, Calif.). All radiolabeled fatty acids were purchased from American Radiolabeled Chemicals Inc. (St. Louis, Mo.). Most other chemicals were purchased from Sigma Chemical Co. (St. Louis, Mo.)

Cell Culture of 3T3-L1 Cells and Differentiation into the Adipoctye Phenotype.

3T3-L1 cells were cultured to confluence in Dulbecco's modified Eagle's medium (DMEM) containing 10% calf serum (CS) by changing the medium every two days as previously described⁽²⁰⁾. Two days after cell confluence, differentiation was initiated by adding differentiation medium (0.5 mM methylisobutylxanthine, 0.25 μM dexamethasone, 1 μg/mL insulin in DMEM containing 10% fetal bovine serum (FBS)). Two days later, methylisobutylxanthine and dexamethasone were removed and insulin (1 μg/mL) was maintained for two more days. Thereafter, cells were grown in DMEM containing 10% FBS in the absence of differentiating reagents by replacing the media every two days. At each time point examined, cells were washed with PBS, removed from cell culture plates by trypsin, washed with medium, centrifuged and washed again with PBS before final pelleting. The cell pellets were collected and stored at −70° C. until future use (typically within one or two weeks).

Lipid Extraction of Differentiated Adipocytes.

Lipids were extracted by a modified Bligh-Dyer technique⁽²¹⁾ utilizing 50 mM LiCl in the aqueous layer in the presence of appropriate internal standards (15:0-15:0 PG, 14:0-14:0 PS, 14:1-14:1 PC, 15:0-15:0 PE, T17:1 TAG)⁽²²⁻²⁵⁾. The internal standards were selected based on their solubility and the lack of any demonstrable endogenous molecular ions in that region which was verified by periodically acquiring mass spectra without internal standards. The lipid extracts were dried under a nitrogen stream, dissolved in chloroform, filtered through 0.2 μm Gelman acrodisc CR PTFE syringe filters, reextracted, and dried under a nitrogen stream. The final lipid residue was resuspended in 1:1 chloroform/methanol prior to ESI/MS analysis.

Electrospray Ionization Mass Spectrometry of Adipocyte Lipid Classes.

ESI mass spectral analysis of lipid molecular species was performed utilizing a Finnigan TSQ Quantum spectrometer (Finnigan MAT, San Jose, Calif.) equipped with an electrospray ion source as previously described⁽²²⁻²⁵⁾. Typically, a 1-min period of signal averaging in the profile mode was employed for each spectrum of the lipid extract. All samples were diluted in 1:1 methanol/chloroform prior to direct infusion into the ESI source using a syringe pump at a rate of 2 μL/min. Anionic phospholipids, except PS in the diluted chloroform extracts of 3T3-L1 cell pellets, were analyzed by ESI-MS in the negative-ion mode and quantified by comparisons of the individual ion peak intensity with internal standard (i.e. 15:0-15:0 PG) after correction for ¹³C isotope effects. PS was analyzed in the neutral loss scanning mode (87 amu) to enhance S/N due to diminutive amounts of PS in differentiating adipocytes. Phosphatidylserines were quantified by comparisons of the individual ion peak intensity with phosphatidylserine internal standard (i.e. 14:0-14:0 PS). Prior to the analyses of choline and ethanolamine glycerophospholipids in the diluted cell extracts, LiOH in methanol (50nmol/mg protein) was added. Choline glycerophospholipids were quantified as their lithium adducts by comparisons with an internal standard (e.g. lithiated 14:1-14:1 PC) after correction for ¹³C isotope effects in the positive ion mode as previously described⁽²⁴⁾. Since TAG massively accumulates during differentiation in adipocytes to overwhelm the amount of choline glycerophospholipids in the cell, it was preferable to separate TAG and choline glycerophospholipids by HPLC as previously described⁽²⁶⁾ prior to ESI/MS analysis. Ethanolamine glycerophospholipids were directly quantified by comparison with an internal standard (e.g. lithiated 15:0-15:0 PE) after correction for ¹³C isotope effects in the negative ion mode as previously described⁽²⁴⁾. Identification of ion peaks was achieved utilizing tandem-MS analyses in the precursor-ion or neutral loss scanning mode^{(25, 27)}. Plasmalogen molecular species were distinguished from alkyl-acyl phospholipid molecular species by treating tissue extracts with acidic vapors prior to mass-spectroscopic analyses as described previously⁽²⁸⁾. TAG molecular species were directly quantitated by neutral loss scanning in the positive-ion mode as previously described⁽²⁵⁾.

Derivatization of Non-esterified Fatty Acids and Fatty Acids in Individual Lipid Classes.

Lipid classes were resolved from the chloroform extract utilizing an Ultrasphere silica HPLC column (4.5×250 mm; 5 μm; Beckman) as the stationary phase by gradient elution at a flow rate of 2 mL/min as described⁽²⁶⁾. Non-esterified fatty acids or fatty acids esterified to individual lipid classes were converted to their corresponding methyl esters by incubation of 1-100 μg lipid in 1 mL of 1 N methanolic HCl at 90° C. for 60 min under nitrogen. After cooling on ice, the mixture was neutralized with Na₂CO₃ and extracted as previously described⁽²⁹⁾. In some cases, monounsaturated fatty acid methyl esters were derivatized with dimethyldisulfide (DMDS) as previously described⁽³⁰⁾. Briefly, to 1-100 μg of fatty acid methyl esters in 0.5 mL hexane were added dimethyldisulfide (0.5 mL) and I₂ (50 μL 60 mg/mL in ethyl ether), and the mixture was incubated at 40° C. in an enclosed tube overnight. Iodine was removed by addition of Na₂S₂O₃ solution (5% in distilled water). The reaction mixture was extracted with hexane, dried over MgSO₄, filtered, dried under nitrogen stream and redissolved in hexane prior to GC/EI/MS analysis⁽³⁰⁾.

Gas Chromatography and Electron Impact Mass Spectrometry of Adipocyte Fatty Acid Methyl Ester.

GC/MS was performed on a Thermoquest SSQ 7000 single stage quadrupole mass spectrometer interfaced with a Varian 3300 gas chromatograph. A fused-silica capillary DB-1 column (12.5 m×0.2 mm I.D., 0.33 μm film thickness) from J&W Scientific (CA, USA) was employed for separation of fatty acid methyl esters. After injection, the column was held at 120° C. for 1 minute prior to increasing the temperature to 270° C. at 10° C./min and kept at 270° C. for 10 additional minutes. For analysis of fatty acid methyl ester DMDS adducts, a DB-17 column (30 m×0.25 mm I.D., 0.251 μm film thickness) was used and the GC oven was maintained at 150° C. for 1 minute prior to increasing the temperature to 280° C. at a rate of 30° C./min and held at 280° C. for an additional 6 minutes. Helium was used as a carrier gas at a constant pressure of 55 kPa. In all cases, the injector and interline temperatures were maintained at 250° C. Electron impact (El) ionization was performed at 70 eV as previously described⁽³¹⁾.

Indirect Immunofluorescent Microscopy

Six days after differentiation 3T3-L1 adipocytes were replated at 30 to 50% of their original density. Adipocytes were fixed for 10 minutes with 2% formaldehyde in phosphate-buffered saline (PBS). The coverslips were rinsed in PBS and then incubated for 1 hour in both PMP70 antiserum (Zymed, San Francisco, Calif. ) diluted 1:500 in microscopy buffer (1% BSA, 0.1% saponin in PBS) and antibody directed against stearoyl-CoA dehydrogenase at 1 μg/mL (Santa Cruz Biotechnology, Santa Cruz, Calif.). The coverslips were rinsed in PBS and then incubated for 30 minutes with Alexa 594 donkey anti-rabbit IgG (Molecular Probes, Eugene, Oreg.) and Alexa 488 Donkey anti-goat IgG (A-11058) both diluted 1:1000 in microscopy buffer.

α-Oxidation of [9,10-³H]-Palmitic Acid in 3T3-L1 Cells During Adipocyte Differentiation.

3T3-L1 cells, at selected times during differentiation, were radiolabeled by incubation with 5 μCi [9,10-³H]-palmitic acid (dissolved in EtOH) injected into the incubation media. After the indicated periods, the media was removed, the cells were washed with PBS, scraped into 1.5 ML of deionized water, and total lipids were extracted according to the method of Bligh-Dyer with 1% acetic acid in the aqueous layer⁽²¹⁾. After addition of 100 μg of each internal standard (C16:0, C15:0, C14:0), the lipids were incubated in 0.4 mL methanol/0.2 mL of 0.3 M HCl at 60° C. for 20 minutes to cleave all vinyl ether linkages. The mixture was continuously incubated at 60° C. for an additional 40 minutes after addition of 1 mL 1 M KOH. The fatty acids were extracted into hexane (2×2 mL) after acidification with of 1 M HCl. The extracted fatty acids were dried under a nitrogen stream, redissolved in 0.4 mL acetone and oxidized with 1 mL 0.1 M KMnO₄/0.1 M H₂SO₄ at 60° C. for 20 minutes. Excess KMnO₄ was removed by addition of Na₂S₂O₃ and the resulting saturated fatty acids were extracted into hexane (2×2 mL) after acidification. Fatty acids were separated by reversed phase HPLC utilizing an octadecyl silica stationary phase (4.6×250 mm, 5 μm particles) with isocratic elution at 2 min/mL employing a mobile phase comprised of methanol/H₂O/H₃PO₄ (85/15/0.1, v/v/v). Column eluents were monitored by UV detection at 210 nm and radioactivity in each saturated fatty acid was quantified by liquid scintillation spectrometry.

[1-¹⁴C]-Palmitate Oxidation Studies.

Measurement of palmitate oxidation was performed as previously described⁽³²⁾. Briefly, 3T3-L1 cells were cultured in T-25 flasks to confluence and induced to differentiate as described above. At selected stages of differentiation, [1-¹⁴C]-palmitate was added to a final concentration of 200 nCi/mL. The flasks were sealed and fitted with a center well comprised of Whatman No. 1 filter paper. At selected time points, the ¹⁴CO₂ liberated from the medium by acidification with 2 mL of 6 M of HCl was collected on filter paper previously alkalinized with 250 μL of 2 M NaOH. Released radiolabeled ¹⁴CO₂ was quantified by liquid scintillation counting.

Reverse Transcription and Quantitative Polymerase Chain Reaction (PCR).

Total RNA was purified from 3T3-L1 cell pellets utilizing a RNeasy® Mini Kit from Qiagen (Valencia, Calif., USA) according to manufacturer's instructions. For cDNA preparation, 250 pmol of random hexamers were hybridized by incubation for 10 min at 25° C. and extended by incubation for 30 min at 48° C. in the presence of 125 units of reverse transcriptase in 100 μL of PCR buffer (5.5 mM MgCl₂, 0.5 mM of each dNTP, and 40 units of RNase inhibitor). Reverse transcriptase was inactivated by incubation at 95° C. for 5 min. Amplification of each target cDNA was performed with TaqMan® PCR reagent kits and quantified by the ABI PRISM 7700 detection system according to the protocol provided by the manufacturer (Applied Biosystems, Foster City, Calif.). A traditionally utilized standard gene, GAPDH, was measured and used as internal standard. Oligonucleotide primer pairs and probes specific for FACO (5′-GG ATGGTAGTCC GGAGAACA/5′-AGTCTGGATCGTTCAGAATC AAG/5′-TCTCGATTTCTCGACGGCGCCG), and PAHX (5′-TGAAGAAAATGGGT TTCTCGTCAT/5′-ACGATCCCTGGTGGTTTCAC/5′-ACTCTGCTCGAAAACGT TGA) were employed.

Protein concentration was determined with a BCA protein assay kit (Pierce, Rockford, Ill.) using bovine serum albumin (BSA) as a standard. All data were normalized to protein content and are presented as the mean±SEM. Statistically significant differences between mean values were determined by unpaired Student's t tests.

Results

Electrospray Ionization Mass Spectrometry of Lipids in Differentiating 3T3-L1 Cells.

Mass spectra of serine and inositol glycerophospholipids during hormone-induced differentiation of 3T3-L1 cells demonstrated the time-dependent accumulation of prominent pseudomolecular ion peaks corresponding to odd chain length fatty acids (denoted by asterisk in FIG. 1, 2). Hormone-induced differentiation of 3T3-L1 cells into adipocytes also resulted in a dramatic shift of the aliphatic chains in inositol and serine glycerophospholipids to lower carbon number molecular species. Both of these features were present in multiple independent preparations (Tables I-V). To identify alterations in ethanolamine and choline glycerophospholipids in adipocytes during differentiation, direct ionization of phospholipids from chloroform extracts coupled with neutral loss scanning of their pseudomolecular ions was employed. Mass spectra of choline glycerophospholipids (FIG. 3) in differentiating adipocytes demonstrated the robust accumulation of odd chain length molecular species as well as a concurrent shortening of aliphatic chain lengths. Similarly, precursor-ion scanning of ethanolamine glycerophospholipids (to clearly visualize each individual molecular species) of day 8 differentiated adipocytes demonstrated accumulation of odd chain length fatty acids and aliphatic chain length shortening (FIG. 4 and Table III). These results were all confirmed by tandem mass spectrometry employing low energy collision-induced dissociation. Collectively, these results demonstrate the accumulation of odd chain length molecular species and aliphatic chain length shortening in all major polar lipid classes of the differentiating adipocyte.

Quantitative neutral loss scanning of lipid extracts from differentiated 3T3-L1 adipocytes demonstrated that TAG molecular species were also highly enriched in C15:0, C15:1, C17:0 and C17:1 acyl moieties (FIG. 5, Table V). To exclude the possibility that odd chain length fatty acids in adipocytes were the result of C15 and C17 fatty acyl moieties in the culture media, direct analysis-of lipids in the nutrient medium was performed. The results demonstrated that <1% of the fatty acids in the media were comprised of odd chain length fatty acids. Importantly, both culture media and cytosol contained both oleic acid and palmitoleic acid.

Identification of the Location of Olefinic Linkage in Odd Chain Length Monounsaturated Fatty Acids in Differentiating 3T3-L1 Adipocytes.

Determination of the molecular species of triglycerides during the differentiation process demonstrated dramatic increases in triglycerides containing unsaturated acyl chains. Remarkably, over 95% of triglyceride molecular species contained at least one unsaturated acyl chain (Table V). Determination of the location of the double bond in the unsaturated odd chain length acyl chains in triglycerides could be used to determine if α oxidation and Δ9 desaturation were random or ordered processes in the sequence of reactions leading to fatty acid storage in the triglyceride compartment of adipocytes. The location of the olefinic linkage in odd chain length fatty acyl moieties would be diagnostic of the order of α oxidation and Δ9 desaturation. For example, if Δ9 desaturation preceded α oxidation in the peroxisomal compartment, then Δ8 unsaturated odd chain length fatty acids would be generated. Similarly, if α oxidation of oleic acid from the medium or cytosol occurred, then Δ8 C17:1 would be produced. Alternatively, if α oxidation of saturated fatty acyl CoAs obligatorily preceded Δ9 desaturation, then only Δ9 olefins would be present in odd chain length moieties in the TAG pool. If these reactions were random (i.e. there was no obligatory order of α oxidation and Δ9 desaturation), adipocytes would contain both Δ8 and Δ9 unsaturated moieties. To this end, I exploited the propensity of olefinic dimethyldisulfide adducts to α fragmentation during collisional activation of their pseudomolecular ions (30). Both C15:1 and C17:1 present in TAG molecular species contained exclusively (>98%) Δ9 desaturated moieties as evidenced by their methylthiol radical induced fragmentation patterns (FIG. 6). These results demonstrate that neither oleic or palmitoleic acid from the medium or cytosol nor SCD desaturated fatty acyl CoAs can undergo α oxidation since Δ8 olefinic linkages were not observed in either C15:1 or C17:1 species. Thus, α oxidation in the peroxisomal compartment must obligatorily precede Δ9 desaturation. Moreover, once α oxidation has occurred, the processed (i.e. a oxidized) fatty acyl CoAs can subsequently undergo Δ9 desaturation and thus be metabolically channeled into TAG synthesis. These results demonstrate that one, and perhaps the major, biochemical mechanism underlying the antiadipogenic effects of SCD I knockouts and knockdowns^{(41, 42)} is the metabolic channeling of saturated fatty acids into oxidative pathways since in the absence of Δ9 desaturation their entry into TAG storage pools is limited (i.e. TAG molecular species contain only diminutive amounts of trisaturated molecular species). Similarly, analysis of odd chain length unsaturated fatty acyl moieties in choline and ethanolamine glycerophospholipids demonstrated the nearly exclusive localization of the double bond at the Δ9 position. Since both culture media and cytosol contained both oleic and palmitoleic acids which possess a double bond exclusively at Δ9 position (FIG. 6E, 6F), the absence of Δ8 fatty acids in differentiating adipocytes demonstrated that unsaturated fatty acids could not be processed by peroxisomal α oxidation. Thus, once the fatty acids are unsaturated, shedding of free energy by α oxidation is not possible and unsaturated fatty acids are channeled toward lipid anabolic pathways.

Recent studies from Hajra and co-workers showed that nearly half of TAG synthesis was initiated via the acyl dihydroxyacetone phosphate pathway within the peroxisomal compartment in differentiated adipocytes⁽³³⁾. The present results suggest the possibility that peroxisomes could contain a Δ9 desaturase activity since Δ9 desaturation must occur after α oxidation and before the first committed step in TAG synthesis. Neither SCD I nor SCD II has previously been identified in the peroxisomal compartment. However, close inspection of the primary sequence of mouse SCD I revealed a putative type II peroxisomal localization sequence at the N terminus (KVKTVPLHL)⁽³⁴⁻³⁶⁾. Further examination of the C terminus of SCD I identified a dilysine transmembrane ER retention signal (KKVS) near the C terminus⁽³⁷⁾. Thus, mouse SCD I has two putative subcellular localization signals. Prior work has demonstrated a type II PTS consensus sequence is sufficient for peroxisome targeting in many cases^{(35, 36)}. However, the large majority of prior studies have assigned SCD I to the ER compartment. Buoyant density gradient centrifugation of hepatic membranes by traditional methods indeed demonstrated that the large majority of SCD I was present in the ER (≈90%). Due to the broad elution of SCD I and the presence of some overlap with peroxisomal markers, the possibility that a small portion of SCD I (≈10%) is present in the peroxisomes could not be excluded (data not shown). To examine this question in adipocytes, immunohistochemistry of differentiated 3T3-L1 adipocytes was performed. A portion of SCD I mass appeared to be overlayed with the peroxisomal marker, PMP70 (FIG. 7). However, caution must be exercised due to the possibility of emission overlap of opposed or stacked structures. I specifically point out that these results do not demonstrate the subcellular compartment where Δ9 desaturation occurs. The data do demonstrate that no Δ9 desaturated fatty acid can be α oxidized in the peroxisomal compartment, thus promoting the chances that Δ9 desaturated fatty acids be recruited into the triglyceride storage pool.

Production of Radiolabeled C15:0 After Incubation of the Differentiating 3T3-L1 Cells With [9,10-³H]-C16:0.

In order to unambiguously demonstrate that the mechanism of production of odd chain length fatty acids in differentiating adipocytes is mediated by α oxidation, initial rate kinetic radiolabeling tests were performed. Incubation of cells with [9,10-³H]-C16:0 for 0.5, 1, 2 or 5 hours resulted in the production of radiolabeled [³H]-C15:0. The rate of α oxidation was approximated by calculating the flux of mass of C16 fatty acids to C15 fatty acids based upon the measured specific activity. For example, cells at day −1, day 4, day 8 were incubated in radiolabeled C16:0 containing media (5 μCi in 4 mL 10% FBS media). Next, cells were harvested at 0.5, 1, 2 and 5 hours and the radioactivity in the C16 pool was measured. Similarly, the mass of C16:0 NEFA in the adipocytes was measured at these time points. From the specific activity of the C16 pool, the rate of α oxidation was calculated to yield an apparent initial rate of conversion of C16 fatty acid to C15 fatty acid of 45 pmol/mg protein/min. The measured rate of α oxidation increased 2 fold in day 4 differentiated cells and 4 fold in day 8 differentiated cells in comparison to resting 3T3-L1 cells (FIG. 8A). I point out that this calculation is an estimate since many other factors including physical or metabolic compartmentation are important in consideration of mass flux. However, I note that by utilizing these basic assumptions, the temporal course of the increase in odd chain length fatty acid mass demonstrated by mass spectroscopy parallels the measured rates of increase of radiolabeled C16:0 to C15:0 conversion.

Fatty Acid Oxidation of [1-¹⁴C]-Palmitate in Differentiating 3T3-L1 Cells.

The rate of total fatty acid oxidation was estimated from measurements of the flux of cells radiolabeled with [1-¹⁴C]-palmitic acid by calculation the specific activity of intracellular palmitic acid at different times after the induction of adipocyte differentiation. From the measurements of radiolabeled C16 NEFA and C16 NEFA mass at 4 and 8 days after differentiation, the specific activity of C16 fatty acid could be calculated and the appearance of ¹⁴CO₂ was used to approximate the overall rate of fatty acid oxidation (both α and β oxidation). Again, I point out that these results represent a first order approximation which does not take into account issues related to compartmentation (either physical or metabolic). Utilizing these approximation, the results demonstrated that fatty acids are oxidized at similar rates in day −1 and day 4 cells but that by day 8 the adipocyte has dramatically increased its oxidative rate (FIG. 8B). Remarkably, the main increase in the oxidative rate of differentiated 3T3-L1 cells is accounted for by the increase in the rate of α oxidation.

Quantitative PCR Analysis of PAHX and FACO RNA Levels.

Since α hydroxylation, which presumably is initiated by PAHX, is the first committed step, and one of the rate determining steps, in odd chain length FA production⁽¹⁵⁾, PAHX mRNA levels were examined after inducing differentiation. As expected, PAHX mRNA level increased four fold during differentiation (FIG. 9A). This result supports the data that straight chain fatty acid α oxidation increases during adipocyte differentiation. Moreover, the mRNA level of FACO, whose protein product catalyzes the rate determining step of peroxisomal fatty acid β oxidation, also dramatically increased during differentiation (FIG. 9B). The proliferation of peroxisomes in conjunction with the entry of their metabolic products into lipid storage pools supports the role of peroxisomes in modulating lipid metabolism during adipocyte differentiation^{(38,39)}.

Discussion

The synthesis and catabolism of fatty acids in mammalian cells have been extensively studied over the last 50 years. The present study utilizes recent advances in lipidomics (reviewed in ⁴⁰) to demonstrate dramatic changes in cellular lipid metabolism that accompany 3T3-L1 cell differentiation into adipocytes after hormone-induced differentiation. Through the detailed account of time-dependent alterations in the individual chemical constituents present in adipocyte cellular membranes and TAG storage pools, new insights into the cellular processing of fatty acids in mammalian cells have been gleaned. The importance of fatty acid α oxidation and the obligatory sequential ordered processing of α oxidation of fatty acids prior to their Δ9 desaturation and subsequent incorporation into TAG molecular species in this murine cell line have been determined.

Prior work has demonstrated the importance of SCD I in both diet and leptin-mediated obesity^{(41, 42)}. For example, SCD I null mice do not become obese in response to high fat diets. Similarly, genetic crosses of SCD I null mice with leptin-deficient mice demonstrate that SCD I deficiency protects against the obesity manifest in leptin-deficient mice. Several clues to the chemical mechanisms underlying these recent important physiologic and genetic observations can be gleaned from careful analysis of the individual molecular species changes that occur during the adipocyte differentiation process. First, molecular species analysis of triglycerides in differentiating adipocytes demonstrates that the overwhelming majority (>95%) of TAG molecular species contain unsaturated fatty acids. Since the aliphatic chains entering the TAG pool are largely derived from saturated fatty acids in the growth media (palmitate and stearate are the most abundant fatty acids in the growth medium) and saturated fatty acids derived from de novo synthetic pathways, it is clear that fatty acid desaturation is an important process in providing the specific building blocks (desaturated fatty acids) used in TAG synthesis. Second, the present results show that saturated fatty acids can be rapidly α oxidized after differentiation (but not in undifferentiated cells) and that mRNAs encoding enzymes involved in peroxisomal fatty acid oxidation are increased. Moreover, in differentiated adipocytes the rate of α oxidation is robust and by first order approximation is likely at least as robust as β oxidation. I specifically point out that the effects of compartmentalization (both physical and metabolic) make these interpretations only approximate. Nonetheless, it seems apparent both by the mass increase of odd chain length fatty acids as well as direct measurements of C16 to C15 conversion that substantial α oxidation of unbranched fatty acids is occurring in the differentiating adipocyte. Third, these results also demonstrate that after α oxidation in the peroxisomes, odd-chain length fatty acids can subsequently be desaturated by Δ9 desaturase. Of note is the demonstration of a putative Type 2 peroxisomal localization sequence in the primary sequence of mouse SCD I. This sequence motif has previously been shown to be sufficient for peroxisomal membrane integration by others⁽³⁵⁾. Both buoyant density gradient centrifugation data and immunohistochemistry were consistent with the notion that a small portion of SCD I resides in the peroxisomal compartment. The desaturase activity of SCD I requires cytochrome b₅ and an electron donor. Previous studies have utilized several different techniques to demonstrate that a portion of cytochrome b₅ is associated with the peroxisomal compartment⁽⁴³⁻⁴⁵⁾. Although unlikely, the results do no exclude the possibility that after α oxidation in the peroxisomal compartment subsequent Δ9 desaturation can occur in the ER if the odd chain length peroxisomal acyl CoA is hydrolyzed, transported to the ER (by either directed or passive diffusion) and subsequently Δ9 desaturated. Wherever the specific anatomic compartment in which Δ9 desaturation occurs, the results collectively demonstrate the functional sequential ordered processes of α oxidation prior to Δ9 desaturation in differentiating adipocytes. In large part, this is due to the inability of unsaturated fatty acids to serve as substrates for α oxidation. Fourth, since odd chain length fatty acids containing Δ9 unsaturated centers are abundant TAG molecular species in differentiated adipocytes, these results underscore the quantitative importance of sequential α oxidation and Δ9 desaturation in generating the aliphatic constituents used to build TAG molecular species. Finally, the close proximity of triglyceride lipid droplets to the peroxisomal compartment in conjunction with the mass spectrometric data raises the intriguing possibility that multiple functionally important interactions exist between peroxisomes and triglyceride droplets^{(38, 39)}. Collectively, these results demonstrate the obligatory ordering of α oxidation and Δ9 desaturation and the importance of peroxisomal lipid metabolism in generating the lipid molecular species present in the lipidome of differentiating adipocytes.

In resting 3T3-L1 cells, only diminutive amounts of odd chain length fatty acids are present and the molecular species distribution in each class of lipids is similar to that found in many other cell types (e.g., inositol glycerophospholipids have predominantly 18:0-20:4 molecular species). However, within days after hormone-induced differentiation, odd-chain length fatty acids began to increase in multiple different lipid classes. Prior work has demonstrated that the peroxisomal enzyme phytanoyl-CoA α hydroxylase can utilize unbranched fatty acids as substrates⁽¹⁵⁾ and the present study demonstrates that PAHX mRNA is increased 4 fold during adipocyte differentiation. Since α hydroxylation is the first committed step, and one of the rate determining steps, in odd chain length FA production^{(13, 15)}, the observed increase in α oxidation is presumably mediated by a transcription-mediated increase in phytanoyl CoA α hydroxylase mRNA, followed by its translation and PAHX catalyzed α oxidation in the peroxisomal compartment. Post translational alterations may also contribute to the observed increase in α oxidation. Early studies have failed to demonstrate the presence of α hydroxylation catalyzed by this enzyme utilizing long chain unbranched fatty acids as substrates⁽¹⁴⁾. However, the recent demonstration of the importance of substrate presentation mediated by sterol carrier protein 2 (and perhaps additional proteins as well in peroxisomes) makes it likely that α oxidation in the peroxisomal compartment is catalyzed by phytanoyl CoA αhydroxylase utilizing SCP 2 or other proteins to facilitate substrate presentation⁽¹⁵⁾. Recent studies suggesting the importance of α oxidation emerged when multiple enzymes involved in the peroxisomal α-hydroxyl fatty acid oxidation have been cloned from both rat and human genomes leading to the expression of protein products with high activity utilizing α-hydroxy unbranched long chain fatty acids (e.g., α-hydroxyl palmitic acid) as substrates⁽⁴⁶⁻⁴⁸⁾.

Since α oxidation utilizes O₂ as a direct electron acceptor, this reaction does not produce any reducing equivalents which can be utilized for ATP synthesis. Thus, α oxidation in the peroxisomal compartment is constitutively uncoupled and serves as a defacto mechanism to shed the Gibbs free energy inherit in the C—C bond of fatty acids (i.e. thermogenesis). Moreover, α oxidation produces CO₂ at the expense of acetyl CoA production and thus does not participate in the efficient conversion of fatty acid into chemical fuel for cellular utilization. Thus, these results indicate a prominent role for fatty acid α oxidation as an important metabolic determinant of the lipid content in the differentiating adipocyte. Collectively, they underscore the role of fatty acid α oxidation in the peroxisomal compartment as a proximal mechanism which modulates fatty acid metabolic fate and energy storage in the differentiating adipocyte.

GC/EI/MS: Gas Chromatography and Electron Impact Mass Spectrometry

PC: phosphatidylcholine

PS: phosphatidylserine

PI: phosphatidylinositol

PE: phosphatidylethanolamine

PG: phosphatidylglycerol

TAG: triacylglycerol

FFA: free fatty acid

Cm:n: fatty acid with m carbons and n double bonds

PTS 2: peroxisome targeting signal 2

DMDS: dimethyl disulfide

SCD: stearoyl-CoA desaturase

SCP 2: sterol carrier protein 2

PAHX: phytanoyl-CoA α hydroxylase

FACO: fatty acyl-CoA oxidase

### REFERENCES

1. Friedman, J. M. (2000) Nature 404, 632-634

2. Kopelman, P. G. (2000) Nature 404, 635-643

3. Gregoire, F. M., Smas, C. M., and Sul, H. S. (1998) Physiol. Rev. 78, 783-809

4. Pi-Sunyer, X. (2003) Science 299, 859-860

5. Spiegelman, B. M., and Flier, J. S. (2001) Cell 104, 531-543

6. Lowell, B. B., and Spiegelman, B. M. (2000) Nature 404, 652-660

7. Green, H., and Kehinde, O. (1976) Cell 7, 105-113

8. Green, H., and Kehinde, O. (1975) Cell 5, 19-27

9. Reginato, M. J., Krakow, S. L., Bailey, S. T., and Lazar, M. A. (1998) J. Biol. Chem. 273, 1855-1858

10. Alexander, M., Curtis, G., Avruch, J., and Goodman, H. M. (1985) J. Biol. Chem. 260, 11978-11985

11. Green, H., and Kehinde, O. (1979) J. Cell. Physiol. 101, 169-171

12. Wanders, R. J. A., Jansen, G. A., and Lloyd, M. D. (2003) Biochim. Biophys. Acta. 1631, 119-135

13. Wanders, R. J. A., Vreken, P., Ferdinandusse, S., Jansen, G. A., Waterman, H. R., Van Roermund, C. W. T., and Van Grunsven E. G. (2001) Biochem. Soc. Trans. 29, 250-267

14. Croes, K., Foulon, V., Casteels, M., Veldhoven, P. P. V., and Mannaerts, G. P. (2000) J. Lipid Res. 41, 629-636

15. Mukherji, M., Kershaw, N. J., Schofield, C. J., Wierzbicki, A. S., and Lloyd, M. D. (2002) Chem. Biol. 9, 597-605

16. Jansen, G. A., van den Brink, D. M., Ofman, R., Draghici, O., Dacremont, G., Wanders, R. J. A. (2001) Biochem. Biophys. Res. Commun. 283, 674-679

17. Jansen, G. A., Ofinan, R., Denis, S., Ferdinandusse, S., Hogenhout, E. M., Jakobs, C., Wanders, R. J. A. (1999) J. Lipid Res. 40, 2244-2254

18. Verhoeven, N. M., and Jakobs, C. (2001) Prog. Lipid Res. 40, 453-466

19. Mihalik, S. J., Morrell, J. C., Kim, D., Sacksteder, K. A., Watkins, P. A., Gould, S. J. (1997) Nat. Genet. 17, 185-189

20. Frost, S. C., and Lane, M. D. (1985) J. Biol. Chem. 260, 2646-2652

21. Bligh, E. G., and Dyer, W. J. (1959) Can. J Biochem. Physiol. 37, 911-917

22. Han, X., and Gross, R. W. (1994) Proc. Natl. Acad. Sci. USA 91, 10635-10639

23. Han, X., Gubitosi-Klug, R. A., Collins, B. J., and Gross, R. W. (1996) Biochemistry 35, 5822-5832

24. Han, X., Abendschein, D. R., Kelly, J. G., and Gross, R. W. (2000) Biochem. J. 352, 79-89

25. Han, X., and Gross, R. W. (2001) Anal. Biochem. 295, 88-100

26. Ford, D. A., Han, X., Homer, C. C., and Gross, R. W. (1996) Biochemistry 35, 7903-7909

27. Han, X., and Gross, R. W. (1995) J. Am. Soc. Mass Spectrom. 6, 1202-1210

28. Ford, D. A., Rosenbloom, K. B., and Gross, R. W. (1992) J. Biol. Chem. 267, 11222-11228

29. Gross, R. W. (1984) Biochemistry 23, 158-165

30. Buser, H.-R., Arn, H., Guerin, P., and Rauscher, S. (1983) Anal. Chem. 55, 818-822

31. Ryhage, R., and Stenhagen, E. (1960) J. Lipid Res. 1, 361-390

32. Gulick, T., Cresci, S., Caira, T., Moore, D. D., and Kelly, D. P. (1994) Proc. Natl. Acad. Sci. USA 91, 11012-11016

33. Hajra, A. K., Larkins, L. K., Das, A. K., Hemati, N., Erickson, R. L., and MacDougald, O. A. (2000) J. Biol. Chem. 275, 9441-9446

34. Ntambi, J. M., Buhrow, S. A., Kaestner, K. H., Christy, R. J., Sibley, E., Kelly, T. J. Jr., and Lane, M. D. (1988) J. Biol. Chem. 263, 17291-17300

35. Rachubinski, R. A., and Subramani, S. (1995) Cell 83, 525-528

36. Subramani, S. (1996) J. Biol. Chem. 271, 32483-32486

37. Teasdale, R. D., Jackson, M. R. (1996) Ann. Rev. Cell Dev. Biol. 12, 27-54

38. Blanchette-Mackie, E. J., Dwyer, N. K., Barber, T., Coxey, R. A., Takeda, T., Rondinone, C. M., Theodorakis, J. L., Greenberg, A. S., and Londos, C. (1995) J. Lipid Res. 36, 1211-1226

39. Novikoff, A. B., Novikoff, P. M., Rosen, O. M., and Rubin, C. S. (1980) J. Cell Biol. 87, 180-196

40. Han, X., and Gross, R. W. (2003) J. Lipid Res. 44, 1071-1079

41. Cohen, P., Miyazaki, M., Socci, N. D., Hagge-Greenberg, A., Liedtke, W., Soukas, A. A., Sharma, R., Hudgins, L. C., Ntambi, J. M., and Friedman, J. M. (2002) Science 297, 240-243

42. Ntambi, J. M., Miyazaki, M., Stoehr, J. P., Lan, H., Kendziorski, C. M., Yandell, B. S., Song, Y., Cohen, P., Friedman, J. M., and Attie, A. D. (2002) Proc. Natl. Acad. Sci. USA 99, 11482-11486

43. Fowler, S., Remacle, J., Trouet, A., Beaufay, H., Berthet, J., Wibo, M., and Hauser, P. (1976) J. Cell Biol. 71, 535-550

44. Appelkvist, E. L., Brunk, U., and Dallner, G. (1981) J. Biochem. Biophys. Methods 5, 203-217

45. Kikuchi, M., Hatano, N., Yokota, S., Shimozawa, N., Imanaka, T., and Taniguchi, H. (2004) J. Biol. Chem. 279, 421-428

46. Belmouden, A., Diep Le, K. H., Lederer, F., and Garchon, H.-J. (1993) Eur. J. Biochem. 214, 17-25

47. Diep Le, K. H., and Lederer, F. (1991) J. Biol. Chem. 266, 20877-20881

48. Jones, J. M., Morrell, J. C., and Gould, S. J. (2000) J. Biol. Chem. 275, 12590-12597

Tables

Table 1: Alterations in Phosphatidylinositol Molecular Species in 3T3-L1 Cells During Differentiation

Phospholipids from 3T3-L1 cells at selected time points during differentiation were extracted by the Bligh and Dyer method. Extracts were analyzed directly by negative-ion ESI/MS as described under “Test Procedures.” Phosphatidylinositol molecular species were quantified in the negative-ion mode by comparisons of the individual ion peak intensities with that of an internal standard (i.e. 15:0/15:0 PG) after correction for ¹³C isotope effects. Collision induced-dissociation was used to confirm each assignment. The results are expressed in nmol/mg of protein and represent means±S.E.M. of at least three independent cultures.

TABLE 1

| Peaks | | | | | | |
|---|---|---|---|---|---|---|
| (m/ | | | | | | |
| z) | Assignment | Day 0 | Day 2 | Day 4 | Day 6 | Day 8 |
| 805.7 | 16:1-16:1 | | | 0.19 ± 0.06 | 0.35 ± 0.03 | 0.33 ± 0.03 |
| 807.7 | 16:0-16:1 | | | 0.20 ± 0.06 | 0.22 ± 0.04 | 0.30 ± 0.07 |
| 819.7 | 16:1-17:1 | | | 0.15 ± 0.02 | 0.43 ± 0.08 | 0.46 ± 0.06 |
| 821.7 | 16:1-17:0/16:0-17:1 | | | 0.19 ± 0.05 | 0.25 ± 0.03 | 0.42 ± 0.04 |
| 831.7 | 16:1-18:2 | | | | 0.16 ± 0.03 | 0.18 ± 0.03 |
| 833.7 | 16:1-18:1 | | | 0.42 ± 0.10 | 0.56 ± 0.07 | 0.56 ± 0.11 |
| 835.7 | 16:0-18:1 | | | 0.29 ± 0.08 | 0.29 ± 0.07 | 0.32 ± 0.02 |
| 843.7 | 17:1-18:3 | | | | 0.13 ± 0.04 | 0.15 ± 0.01 |
| 845.7 | 17:1-18:2 | | | | 0.20 ± 0.06 | 0.25 ± 0.05 |
| 847.7 | 17:1-18:1 | | | 0.17 ± 0.03 | 0.33 ± 0.10 | 0.33 ± 0.06 |
| 855.7 | 16:1-20:4 | | | 0.18 ± 0.04 | 0.40 ± 0.06 | 0.39 ± 0.05 |
| 857.7 | 16:0-20:4 | | | 0.41 ± 0.05 | 0.79 ± 0.09 | 0.90 ± 0.19 |
| 859.7 | 16:0-20:3 | | | 0.25 ± 0.05 | 0.33 ± 0.04 | 0.37 ± 0.07 |
| 861.7 | 18:1-18:1 | | | 0.33 ± 0.07 | 0.25 ± 0.04 | 0.21 ± 0.04 |
| 869.7 | 17:1-20:4 | | | 0.12 ± 0.02 | 0.34 ± 0.05 | 0.46 ± 0.09 |
| 871.7 | 17:0-20:4 | | | 0.16 ± 0.04 | 0.39 ± 0.05 | 0.56 ± 0.11 |
| 873.7 | 17:0-20:3 | | | 0.10 ± 0.01 | 0.15 ± 0.01 | 0.19 ± 0.03 |
| 881.7 | 18:2-20:4 | 0.23 ± 0.06 | 0.20 ± 0.02 | 0.14 ± 0.04 | 0.21 ± 0.04 | 0.19 ± 0.03 |
| 883.7 | 18:1-20:4 | 1.05 ± 0.20 | 1.00 ± 0.05 | 0.64 ± 0.10 | 0.72 ± 0.11 | 0.59 ± 0.11 |
| 885.7 | 18:0-20:4 | 1.38 ± 0.25 | 1.58 ± 0.09 | 1.14 ± 0.08 | 0.86 ± 0.10 | 0.51 ± 0.09 |
| 887.7 | 18:0-20:3 | 0.54 ± 0.09 | 0.51 ± 0.04 | 0.34 ± 0.05 | 0.24 ± 0.04 | 0.13 ± 0.03 |
| | Total | 3.19 ± 0.58 | 3.29 ± 0.15 | 5.42 ± 0.91 | 7.60 ± 1.1 | 7.78 ± 1.2 |

Table 2: Alterations in Phosphatidylserine Molecular Species in 3T3-L1 Cells During Differentiation.

Phospholipids from 3T3-L1 cells at selected time points during differentiation were extracted by the Bligh and Dyer method and analyzed by negative-ion ESI/MS/MS as described under “Test Procedures.” Phosphatidylserine molecular species were quantified in the negative-ion mode by ESI/MS/MS profiling by neutral loss scanning of 87 amu by comparisons of the individual ion peak intensities with that of an internal standard (i.e. 14:0/14:0 PS), after correction for ¹³C isotope effects. The results are expressed in nmol/mg of protein and represent means±S.E.M. of at least three independent cultures.

TABLE 2

| Peaks | | | | | | |
|---|---|---|---|---|---|---|
| (m/z) | Assignment | Day 0 | Day 2 | Day 4 | Day 6 | Day 8 |
| 732.6 | 16:0-16:1 | | | | 0.45 ± 0.09 | 0.45 ± 0.02 |
| 746.6 | 16:1-17:0/16:0-17:1 | | | 0.37 ± 0.04 | 0.78 ± 0.22 | 1.83 ± 0.37 |
| 758.6 | 16:1-18:1 | | | | 0.35 ± 0.06 | 0.37 ± 0.10 |
| 760.6 | 16:1-18:0/16:0-18:1 | | | 1.46 ± 0.11 | 1.54 ± 0.18 | 2.28 ± 0.44 |
| 772.6 | 17:1-18:1 | | | | 0.16 ± 0.03 | 0.29 ± 0.15 |
| 774.6 | 17:0-18:1/17:1-18:0 | | | 0.34 ± 0.05 | 0.51 ± 0.15 | 0.80 ± 0.24 |
| 786.6 | 18:1-18:1 | 0.76 ± 0.10 | 0.72 ± 0.16 | 0.78 ± 0.04 | 0.47 ± 0.10 | 0.37 ± 0.18 |
| 788.6 | 18:0-18:1 | 1.41 ± 0.27 | 2.18 ± 0.28 | 2.42 ± 0.26 | 1.52 ± 0.19 | 0.80 ± 0.30 |
| 808.7 | 18:1-20:4 | 0.36 ± 0.10 | 0.12 ± 0.00 | 0.14 ± 0.06 | 0.10 ± 0.07 | 0.06 ± 0.01 |
| 810.7 | 18:0-20:4 | 0.49 ± 0.05 | 0.34 ± 0.12 | 0.39 ± 0.13 | 0.41 ± 0.10 | 0.31 ± 0.14 |
| 812.7 | 18:0-20:3 | 0.68 ± 0.31 | 0.39 ± 0.14 | 0.48 ± 0.05 | 0.30 ± 0.07 | 0.22 ± 0.12 |
| 834.7 | 18:0-22:6 | 0.19 ± 0.07 | 0.19 ± 0.10 | 0.20 ± 0.01 | | |
| 836.7 | 18:1-22:6 | 0.43 ± 0.06 | 0.39 ± 0.10 | 0.62 ± 0.02 | 0.33 ± 0.05 | 0.16 ± 0.07 |
| 838.7 | 18:2-22:6 | | 0.13 ± 0.01 | 0.10 ± 0.04 | 0.10 ± 0.11 | 0.11 ± 0.10 |
| | Total | 4.3 ± 0.7 | 4.5 ± 0.6 | 7.3 ± 0.5 | 7.0 ± 0.9 | 8.1 ± 1.9 |

Table 3: Alterations in Ethanolamine Glycerophospholipid Molecular Species in 3T3-L1 Cells During Differentiation.

Phospholipids from 3T3-L1 cells at selected time points during differentiation were extracted by the Bligh and Dyer method as described under “Test Procedures.” Ethanolamine glycerophospholipids were analyzed in the negative-ion mode after addition of LiOH (50 nmol/mg protein) and quantified by comparisons of the individual ion peak intensities with an internal standard (i.e. 15:0/15:0 PE) after correction for ¹³C isotope effects. The results are expressed in nmol/mg of protein and represent means±S.E.M. of at least three independent cultures. D (diacyl) and P (plasmenyl) indicate phosphatidylethanolamine and plasmenylethanolamine molecular species, respectively.

TABLE 3

| Peaks | | | | | | |
|---|---|---|---|---|---|---|
| (m/z) | Assignment | Day 0 | Day 2 | Day 4 | Day 6 | Day 8 |
| 672.6 | P16:0-16:1 | 0.4 ± 0.1 | 0.9 ± 0.2 | 1.8 ± 0.1 | 1.9 ± 0.5 | 3.2 ± 0.5 |
| 686.6 | D16:1-16:1 | 0.7 ± 0.3 | 1.2 ± 0.1 | 5.0 ± 0.3 | 7.0 ± 0.5 | 9.7 ± 0.4 |
| 688.6 | D16:0-16:1 | 0.5 ± 0.1 | 0.4 ± 0.1 | 7.4 ± 0.1 | 7.5 ± 0.6 | 9.2 ± 0.2 |
| 698.6 | P18:1-16:1 | 0.6 ± 0.1 | 0.7 ± 0.1 | 1.8 ± 0.1 | 1.7 ± 0.1 | 2.1 ± 0.3 |
| 700.6 | P16:0-18:1/D16:1-17:1 | 1.5 ± 0.4 | 1.9 ± 0.2 | 3.6 ± 0.2 | 4.5 ± 0.3 | 7.2 ± 0.1 |
| 702.6 | P16:0-18:0/D16:0-17:1/16:1-17:0 | 1.7 ± 0.6 | 1.0 ± 0.3 | 2.9 ± 0.5 | 5.3 ± 0.4 | 7.5 ± 1.9 |
| 712.6 | P18:1-17:1 | 0.5 ± 0.1 | 0.6 ± 0.1 | 1.1 ± 0.1 | 1.0 ± 0.6 | 1.8 ± 0.1 |
| 714.6 | D16:1-18:1/17:1-17:1 | 0.8 ± 0.1 | 1.0 ± 0.1 | 6.5 ± 0.2 | 6.0 ± 0.3 | 7.4 ± 0.3 |
| 716.6 | D16:0-18:1/17:0-17:1 | 1.2 ± 0.1 | 2.1 ± 0.2 | 6.3 ± 0.3 | 4.8 ± 0.2 | 6.3 ± 0.2 |
| 722.6 | P16:0-20:4 | 1.2 ± 0.1 | 2.2 ± 0.2 | 4.1 ± 0.1 | 3.4 ± 0.1 | 3.2 ± 0.4 |
| 726.6 | P18:1-18:1 | 1.5 ± 0.1 | 2.3 ± 0.1 | 2.4 ± 0.3 | 1.6 ± 0.2 | 1.5 ± 0.2 |
| 728.6 | P18:0-18:1/D18:1-18:1 | 1.9 ± 0.1 | 1.6 ± 0.1 | 2.2 ± 0.3 | 2.8 ± 0.2 | 3.9 ± 0.6 |
| 730.6 | D17:0-18:1/17:1-18:0 | 0.8 ± 0.1 | 0.8 ± 0.1 | 1.4 ± 0.3 | 1.8 ± 0.1 | 3.5 ± 0.5 |
| 738.6 | D16:0-20:4 | 1.1 ± 0.2 | 0.9 ± 0.1 | 2.7 ± 0.2 | 3.6 ± 0.2 | 4.8 ± 0.7 |
| 740.6 | D16:0-20:3 | 1.3 ± 0.2 | 1.1 ± 0.1 | 1.4 ± 0.4 | 1.4 ± 0.2 | 1.8 ± 0.1 |
| 742.6 | D18:1-18:1 | 2.9 ± 0.1 | 2.3 ± 0.1 | 2.7 ± 0.2 | 2.0 ± 0.1 | 2.0 ± 0.2 |
| 744.6 | D18:0-18:1 | 2.5 ± 0.3 | 3.4 ± 0.2 | 2.4 ± 0.1 | 1.5 ± 0.2 | 2.3 ± 0.4 |
| 746.6 | D18:0-18:0 | 2.2 ± 0.2 | 2.9 ± 0.1 | 3.1 ± 0.2 | 1.8 ± 0.2 | 2.1 ± 0.3 |
| 748.6 | P18:1-20:4 | 3.6 ± 0.1 | 5.6 ± 0.3 | 6.4 ± 0.5 | 4.1 ± 0.3 | 2.6 ± 0.3 |
| 750.6 | P18:0-20:4 | 2.5 ± 0.1 | 3.6 ± 0.2 | 4.5 ± 1.2 | 3.0 ± 0.2 | 2.8 ± 0.2 |
| 752.6 | D17:0-20:4 | 1.4 ± 0.1 | 1.6 ± 0.1 | 1.9 ± 0.1 | 3.5 ± 0.2 | 5.4 ± 0.4 |
| 762.6 | D16:0-22:6 | 2.2 ± 0.2 | 0.9 ± 0.1 | 1.6 ± 0.4 | 1.7 ± 0.1 | 1.9 ± 0.3 |
| 764.6 | D18:1-20:4 | 3.9 ± 0.0 | 1.8 ± 0.1 | 2.9 ± 0.2 | 2.8 ± 0.1 | 2.3 ± 0.9 |
| 766.6 | D18:0-20:4 | 3.0 ± 0.2 | 2.4 ± 0.2 | 3.5 ± 0.2 | 4.0 ± 0.1 | 4.0 ± 0.3 |
| 774.6 | P18:0-22:6 | 1.4 ± 0.1 | 2.0 ± 0.2 | 1.7 ± 0.1 | 1.0 ± 0.1 | 2.1 ± 1.2 |
| 788.7 | D18:1-22:6 | 1.1 ± 0.1 | 0.7 ± 0.1 | 1.0 ± 0.2 | 1.1 ± 0.1 | 1.4 ± 0.2 |
| 790.7 | D18:0-22:6 | 1.6 ± 0.1 | 1.4 ± 0.1 | 1.6 ± 0.1 | 1.3 ± 0.1 | 1.1 ± 0.2 |
| 792.7 | D18:0-22:5 | 2.2 ± 0.2 | 2.1 ± 0.1 | 1.6 ± 0.1 | 1.0 ± 0.1 | 0.9 ± 0.1 |
| 794.7 | D18:0-22:4 | 1.9 ± 0.7 | 2.1 ± 0.1 | 0.7 ± 0.1 | 0.7 ± 0.1 | 1.1 ± 0.3 |
| | Total | 48.2 ± 2.3 | 51.3 ± 3.2 | 86.0 ± 3.4 | 83.9 ± 3.3 | 105.1 ± 5.3 |

Table 4: Alterations in Phosphatidylcholine Molecular Species in 3T3-L1 Cells During Differentiation.

Phospholipids from 3T3-L1 cells at selected time points during differentiation were extracted by the Bligh and Dyer method as described under “Test Procedures.” Phosphatidylcholines were purified by straight phase HPLC as described (26) and analyzed as their Li⁺ adducts in the positive-ion mode after addition of LiOH (50 nmol/mg protein). Phosphatidylcholine molecular species were quantified by comparisons of the individual ion peak intensities with that of an internal standard (i.e. 14:1/14:1 PC), after correction for ¹³C isotope effects. The results are expressed in nmol/mg of protein and represent means±S.E.M. of at least three independent cultures.

TABLE 4

| Peaks | | | | | | |
|---|---|---|---|---|---|---|
| (m/z) | Assignment | Day 0 | Day 2 | Day 4 | Day 6 | Day 8 |
| 724.6 | 15:0-16:1 | | | 3.2 ± 0.5 | 6.9 ± 0.4 | 8.8 ± 0.9 |
| 736.6 | 16:1-16:1 | 1.5 ± 0.2 | 2.1 ± 0.2 | 6.0 ± 1.0 | 8.5 ± 0.3 | 10.1 ± 1.2 |
| 738.6 | 16:0-16:1 | 3.1 ± 0.2 | 7.0 ± 0.2 | 13.9 ± 1.4 | 15.6 ± 0.9 | 20.3 ± 2.1 |
| 740.6 | 16:0-16:0 | 2.9 ± 0.3 | 4.6 ± 0.3 | 3.2 ± 0.6 | 2.5 ± 0.4 | 2.7 ± 0.2 |
| 750.6 | 16:1-17:1 | | | 2.1 ± 0.3 | 3.9 ± 0.2 | 6.2 ± 0.5 |
| 752.6 | 16:0-17:1/16:1-17:0 | | | 4.4 ± 0.8 | 7.7 ± 0.5 | 13.2 ± 1.1 |
| 754.6 | 16:0-17:0 | | | 1.6 ± 0.5 | 2.8 ± 0.8 | 3.9 ± 0.4 |
| 764.6 | 16:1-18:1 | 3.5 ± 0.3 | 6.2 ± 0.2 | 6.3 ± 0.8 | 5.8 ± 0.4 | 6.7 ± 0.6 |
| 766.6 | 16:1-18:0/16:0-18:1 | 6.6 ± 0.5 | 12.1 ± 0.5 | 9.1 ± 1.1 | 6.6 ± 0.4 | 7.8 ± 0.7 |
| 768.6 | 16:0-18:0 | 1.3 ± 0.3 | 1.1 ± 0.1 | 0.9 ± 0.2 | 1.5 ± 0.6 | 2.8 ± 0.4 |
| 778.6 | 17:1-18:1 | | 2.2 ± 0.2 | 1.8 ± 0.2 | 1.6 ± 0.2 | 2.3 ± 0.6 |
| 780.6 | 17:1-18:0/17:0-18:1 | | 1.8 ± 0.1 | 1.3 ± 0.3 | 1.6 ± 0.1 | 2.6 ± 0.4 |
| 782.6 | 17:0-18:0 | | 1.6 ± 0.2 | 0.8 ± 0.2 | 0.8 ± 0.3 | 1.3 ± 0.5 |
| 786.7 | 16:1-20:4 | 1.7 ± 0.3 | 1.9 ± 0.1 | 1.5 ± 0.2 | 0.9 ± 0.1 | 1.1 ± 0.1 |
| 788.7 | 16:0-20:4 | 1.7 ± 0.2 | 2.6 ± 0.1 | 1.6 ± 0.2 | 1.1 ± 0.2 | 1.0 ± 0.2 |
| 790.7 | 18:1-18:2 | 2.6 ± 0.3 | 4.2 ± 0.2 | 1.9 ± 0.1 | 1.2 ± 0.1 | 1.1 ± 0.1 |
| 792.7 | 18:1-18:1 | 5.6 ± 0.4 | 6.6 ± 0.1 | 3.2 ± 0.4 | 1.4 ± 0.2 | 1.2 ± 0.2 |
| 794.7 | 18:0-18:1 | 3.2 ± 0.6 | 3.7 ± 0.1 | 3.1 ± 0.4 | 1.5 ± 0.1 | 1.0 ± 0.1 |
| 812.7 | 18:2-20:4 | 2.0 ± 0.5 | 1.6 ± 0.0 | 1.1 ± 0.1 | 0.5 ± 0.1 | 0.7 ± 0.2 |
| 814.7 | 18:1-20:4 | 2.0 ± 0.1 | 2.3 ± 0.3 | 1.1 ± 0.1 | 0.7 ± 0.1 | 0.4 ± 0.1 |
| 816.7 | 18:0-20:4 | 1.8 ± 0.3 | 2.6 ± 0.1 | 1.3 ± 0.1 | 0.7 ± 0.2 | 0.7 ± 0.1 |
| 818.7 | 18:0-20:3 | 1.7 ± 0.3 | 2.0 ± 0.1 | 1.5 ± 0.2 | 0.9 ± 0.3 | 1.2 ± 0.1 |
| | Total | 41.1 ± 4.4 | 66.0 ± 3.0 | 71.0 ± 7.6 | 74.7 ± 4.3 | 96.9 ± 9.0 |

Table 5: Alterations in Triglycerides Molecular Species in 3T3-L1 Cells During Differentiation.

Triglycerides from 3T3-L1 cells at selected time points during differentiation were extracted by the Bligh and Dyer method and analyzed as their Li⁺ adducts directly by positive-ion ESI/MS/MS with neutral loss profiling of all possible fatty acids in 3T3-L1 cells as previously described⁽²⁵⁾. Ion abundance for all peaks during tandem mass spectrometry was acquired and corrected for ¹³C isotope effects and the sensitivity factors for each of the parent ions as described under “Test Procedures.” Triglyceride molecular species were quantified by comparisons of the total ion counts of individual molecular species with that of an internal standard (i.e. T17:1 TAG). The results are expressed in nmol/mg of protein and represent means±S.E.M. of at least three independent cultures.

TABLE 5

| Peaks | | | | | |
|---|---|---|---|---|---|
| (m/z) | Assignment | Day 2 | Day 4 | Day 6 | Day 8 |
| 753.6 | 14:1/15:0/15:1 | | | 2.9 ± 0.7 | 18.0 ± 5.9 |
| 755.6 | 14:1/15:0/15:0 | | | 2.7 ± 0.5 | 16.7 ± 3.9 |
| 755.6 | 14:0/15:0/15:1 | | 1.4 ± 0.4 | 1.8 ± 0.3 | 11.1 ± 2.6 |
| 757.6 | 14:015:0/15:0 | | | 1.0 ± 0.2 | 8.5 ± 0.2 |
| 765.6 | 14:1/15:1/16:1 | | | 2.8 ± 0.5 | 15.4 ± 2.3 |
| 767.6 | 14:1/15:0/16:1 | | 2.0 ± 0.6 | 13.8 ± 1.2 | 69.9 ± 12.7 |
| 769.6 | 14:0/15:0/16:1 | | 4.9 ± 1.3 | 12.0 ± 1.4 | 43.7 ± 6.7 |
| 769.6 | 14:0/15:1/16:0 | | | 3.8 ± 0.4 | 26.2 ± 4.0 |
| 769.6 | 14:1/15:0/16:0 | | 0.5 ± 0.1 | 1.6 ± 0.2 | 17.5 ± 2.7 |
| 771.6 | 14:0/15:0/16:0 | | | 3.1 ± 0.7 | 21.2 ± 4.9 |
| 771.6 | 15:0/15:0/15:0 | | | | 5.3 ± 1.2 |
| 779.6 | 15:1/15:1/16:1 | | 3.4 ± 1.4 | 9.7 ± 1.7 | 44.6 ± 7.6 |
| 781.6 | 15:0/15:1/16:1 | | 5.9 ± 1.3 | 20.2 ± 2.6 | 92.7 ± 14.0 |
| 781.6 | 15:0/15:1/16:1 | | 6.6 ± 1.5 | 17.2 ± 2.2 | 64.4 ± 9.7 |
| 783.6 | 15:0/15:0/16:1 | | 4.6 ± 0.6 | 20.5 ± 2.8 | 106.7 ± 12.2 |
| 783.6 | 14:0/16:0/16:1 | | 11.2 ± 1.4 | 16.7 ± 2.3 | 62.3 ± 7.1 |
| 783.6 | 14:1/16:0/16:0 | | 1.0 ± 0.1 | 2.3 ± 0.3 | 8.9 ± 1.0 |
| 785.6 | 15:0/15:0/16:0 | | 3.9 ± 0.7 | 4.4 ± 1.5 | 24.3 ± 1.1 |
| 785.6 | 14:0/16:0/16:0 | | 1.3 ± 0.2 | 1.8 ± 0.6 | 10.9 ± 0.5 |
| 793.6 | 16:1/16:1/15:1 | | 3.0 ± 0.5 | 10.8 ± 2.1 | 47.2 ± 4.1 |
| 793.6 | 15:1/15:1/17:1 | | | 2.9 ± 0.6 | 14.9 ± 1.3 |
| 795.6 | 16:1/16:1/15:0 | | 15.1 ± 3.1 | 61.1 ± 7.0 | 258.3 ± 16.6 |
| 797.7 | 16:0/16:1/15:0 | | 16.5 ± 2.5 | 51.6 ± 6.3 | 199.7 ± 23.4 |
| 799.7 | 16:0/16:0/15:0 | | 4.5 ± 0.7 | 7.2 ± 1.9 | 28.8 ± 5.1 |
| 807.7 | 16:1/16:1/16:1 | 0.9 ± 0.4 | 14.7 ± 4.4 | 42.6 ± 7.3 | 132.2 ± 10.1 |
| 809.7 | 16:1/16:1/16:0 | 2.2 ± 0.8 | 34.0 ± 5.0 | 88.6 ± 14.8 | 270.3 ± 15.3 |
| 811.7 | 16:0/16:0/16:1 | 1.8 ± 0.3 | 27.5 ± 1.5 | 39.6 ± 5.1 | 56.9 ± 5.8 |
| 811.7 | 15:0/16:1/17:0 | | | 9.1 ± 1.2 | 67.0 ± 6.8 |
| 811.7 | 15:0/16:0/17:1 | | | 5.3 ± 0.7 | 41.9 ± 4.3 |
| 813.7 | 16:0/16:0/16:0 | 0.5 ± 0.5 | 6.4 ± 0.2 | 6.2 ± 2.0 | 23.8 ± 4.9 |
| 821.7 | 16:1/16:1/17:1 | 0.4 ± 0.5 | 7.5 ± 2.2 | 25.3 ± 2.9 | 86.0 ± 2.4 |
| 823.7 | 16:0/16:1/17:1 | | 11.5 ± 1.5 | 28.6 ± 2.7 | 98.0 ± 2.0 |
| 823.7 | 16:1/16:1/17:0 | 0.9 ± 0.4 | 4.9 ± 0.6 | 16.8 ± 1.6 | 46.1 ± 0.9 |
| 825.7 | 16:0/16:0/17:1 | | 1.8 ± 0.1 | 4.7 ± 1.6 | 16.5 ± 1.1 |
| 825.7 | 16:0/16:1/17:0 | | 10.9 ± 0.8 | 15.1 ± 5.1 | 46.6 ± 3.2 |
| 825.7 | 15:0/16:0/18:1 | | | 6.2 ± 2.1 | 12.0 ± 0.8 |
| 827.7 | 16:0/16:0/17:0 | | 4.2 ± 0.6 | 4.6 ± 1.7 | 10.4 ± 3.2 |
| 835.7 | 16:1/16:1/18:1 | | 13.1 ± 2.7 | 24.4 ± 2.3 | 35.2 ± 2.9 |
| 835.7 | 16:1/17:1/17:1 | | 2.3 ± 0.5 | 7.3 ± 0.7 | 43.0 ± 3.5 |
| 837.7 | 16:0/16:1/18:1 | 1.0 ± 0.2 | 13.2 ± 1.2 | 26.0 ± 3.6 | 45.7 ± 6.6 |
| 837.7 | 16:1/17:1/17:0 | | 3.7 ± 0.3 | 5.7 ± 0.8 | 20.6 ± 3.0 |
| 839.7 | 16:0/16:0/18:1 | 1.4 ± 1.1 | 11.4 ± 0.5 | 9.9 ± 1.5 | 12.3 ± 2.3 |
| 839.7 | 16:0/17:0/17:1 | | | 4.4 ± 0.7 | 13.5 ± 2.6 |
| 851.7 | 16:1/17:0/18:1 | | 2.7 ± 0.3 | 11.1 ± 1.4 | 15.8 ± 0.9 |
| | Total | 9.1 ± 3.7 | 255.5 ± 25.9 | 653.1 ± 88.4 | 2311.3 ± 160.5 |

In an aspect, a method of TAG analysis and lipid analysis useful in this discovery is carried out by using a method disclosed in U.S. patent application Ser. No. 10/606,601 filed Jun. 26, 2003, “Spectrometric Quantitation of Triglyceride Molecular Species” now Publication 2004-0063118 published Apr. 1, 2004 the contents of which is incorporated herein by reference in their entirety. A level expression greater or less than expression of odd chain length fatty acids in an absence of the substance selected to be measured indicates and is determinant of activity in modulating adipocyte α oxidation and thermogenesis.

In a first embodiment, in regard to the method of analysis disclosed in U.S. Ser. No. 10/606,601, a method for the determination of TG individual (i.e. seperate) molecular species in a composition of matter such as the above in a biological sample comprises subjecting the biological sample to lipid extraction to obtain a lipid extract and subjecting the lipid extract to electrospray ionization tandem mass spectrometry (ESI/MS/MS) providing TG molecular species composition as a useful output determination with special reference to the abundance of odd chain length fatty acids and overall TAG mass.

In an aspect, the inventive concept comprises analyzing a biological sample using electrospray ionization tandem mass spectrometry (ESI/MS/MS) and performing a two dimensional analysis with cross peak contour analysis on the output of the ESI/MS/MS to provide a fingerprint of triglyceride individual (i.e. separate) molecular species.

Briefly, the inventive methods present a novel approach/method which quantitates individual molecular species of triglycerides by two dimensional electrospray ionization mass spectroscopy with neutral loss scanning. This method is also useful for polar lipid analysis by ESI/MS using conditions as outlined in U.S. Ser. No. 10/606,601. This method provides a facile way to fingerprint each patient's (or biologic samples) triglyceride composition of matter (individual molecular species content) and lipid composition of matter directly from chloroform extracts of biologic samples. Through selective ionization and subsequent deconvolution of 2D intercept density contours of the pseudomolecular parent ions and their neutral loss products, the individual molecular species of triglycerides and phospholipids can be determined directly from chloroform extracts of biological material. This 2D (two dimensional) approach comprises a novel enhanced successful functional therapy model for the automated determination and global fingerprinting of each patient's serum or cellular triglyceride and phospholipid profile content thus providing the facile determination of detailed aspects of lipid metabolism underlying disease states and their response to diet, exercise or drug therapy.

In an aspect of this inventive method, tandem mass spectroscopic separation of specific lipid class-reagent ion pairs is used in conjunction with contour density deconvolution of cross peaks resulting from neutral losses of aliphatic chains to determine the individual triglyceride molecular species from a biological sample (blood, liver, muscle, feces, urine, tissue biopsy, or rat myocardium.).

In an aspect, a biological sample is processed in tandem mass spectrometer a first mass spectrometer set up in a tandem arrangement with another mass spectrometer. In that regard the biological sample can be considered as sorted and weighed in the first mass spectrometer, then broken into parts in an inter-mass spectrometer collision cell, and a part or parts of the biological sample are thereafter sorted and weighed in the second mass spectrometer thereby providing a mass spectrometric output readily and directly useable from the tandem mass spectrometer.

In an aspect, a pre-analysis separation comprises a separation of lipoproteins prior to lipid extraction. In an aspect, the pre-analysis separation comprises at least one operation or process which is useful to provide an enhanced biological sample to the electrospray ionization tandem mass spectrometry (ESI/MS/MS). In an aspect, a pre-analysis separation is performed on a biological sample and two compositions are prepared accordingly from the biological sample. In an aspect one composition comprises high density lipoproteins and another composition comprises low density lipoproteins and variants thereof comprised of intermediate densities which can, if necessary, be resolved by chromatographic or other density techniques.

Generally, a biological sample taken is representative of the subject from which or of which the sample is taken so that an analysis of the sample is representative of the subject. In an aspect a representative number of samples are taken and analyzed of a subject such that a recognized and accepted statistical analysis indicates that the analytic results are statistically valid. Typically the composition is aqueous based and contains proteinaceous matter along with triglycerides. For example, a human blood sample is sometimes used. Through use of this inventive method, a plasma sample can be analyzed and appropriate information from the plasma can be extracted in a few minutes. Alternatively, information can be taken from the cells in the blood as well.

In an aspect, serum is utilized as a biological sample. After whole blood is removed from a human body and the blood clots outside the body, blood cells and some of the proteins become solid leaving a residual liquid which is serum.

In an aspect a control sample is employed in the analysis.

In an aspect, the biological sample or a representative aliquot or portion thereof is subjected to lipid extraction to obtain a lipid extract suitable for ESI/MS/MS. In an aspect lipids are extracted from the sample which in an aspect contains a tissue matrix. Non-lipid contaminants should be removed from the lipid extract.

In one aspect lipid extraction is carried by the known lipid extraction process of Folch as well as by the known lipid extraction process of Bligh and Dyer. These useful lipid extraction process are described in Christie, W. W. Preparation of lipid extracts from tissues. In: Advances in Lipid Methodology—Two, pp. 195-213 (1993) (edited by W. W. Christie, Oily Press, Dundee) EXTRACTION OF LIPIDS FROM SAMPLES William W. Christie The Scottish Crop Research Institute, Invergowrie, Dundee DD2 5DA, Scotland all of which are incorporated herein in their entirety by reference. The useful Folch extraction process is reported at Folch et al. (1957) J. Biol. Chem. 226, 497-509 which is incorporated herein in its entirety by reference.

Generally, lipid extraction is carried out very soon in time on the tissue matrix or immediately after removal (harvest) of tissues (tissue matrix) from humanely sacrificed organisms which have been living (carried out using and following acceptable animal welfare protocols). Alternatively, tissues are stored in such a way that they are conservatively preserved for future use. In an aspect, a lipid extract is provided and used to produce ionized atoms and molecules in the inventive analytical method as feed to the ESI mass spectrometer in our novel analysis method.

In an aspect a chloroform lipid extract is employed as a lipid extract composition fed to the ESI mass spectrometer. The effluent from the ESI is fed to a tandem mass spectrometer (i.e. from the exit of the ESI).

In an aspect, a Freezer Mill 6800 from Fisher Bioblock Scientific is used to finely pulverize soft or hard harvested tissues of a biological sample in one or two minutes in liquid nitrogen to render the tissue sufficiently pliable and porous for lipid extraction. Alternatively, the pulverization of the harvested tissue is carried out by subjecting the harvested tissue to hand directed mashing and pulverization using a hand directed stainless-steel mortar and pestle. In a further aspect, an enzymatic digestion is carried out on the harvested tissue which is harvested from a preserved cadaver.

In an aspect, lipids are contained in the lipid extract following the lipid extraction. Generally the extraction is a suitable liquid/liquid or liquid/solid extraction whereby the TG are contained in the extract. In an aspect the extractant has sufficient solvating capability power and solvating capacity so as to solvate a substantial portion of the TG therein or substantially all of the TG present in the biological sample and is contained in the lipid extract.

In an aspect, chloroform is employed as an extractant to produce a useful lipid extract. Other useful extractants include but are not limited to those extractants which have a solvating power, capability and efficiency substantially that of chloroform with regard to the TG molecular species.

The inventive process creates charged forms of very high molecular weight TG molecules obtained via lipid extraction of a biological sample as a part of the process of detecting and analyzing biological samples containing TG.

In an aspect, in order to detect for and analyze ionized atoms and molecules such as TG molecular species in a biological sample, the lipid extract of that biological sample is used to produce ionized atoms and molecules by an ionization method such as electrospray ionization (ESI). As used herein, the term ESI includes both conventional and pneumatically-assisted electrospray mass spectrometry.

In use, the inventive procedure operates by producing droplets of a sample composition by pneumatic nebulization which compresses and forces a biological sample composition containing TG such as an analyte containing TG into a proximal end of a mechanical means housing or holding a fine sized orifice such as a needle or capillary exiting at the distal end of the orifice at which there is applied a sufficient potential. Generally the orifice is a very small bore full length orifice having an internal average diameter or bore in the range from about 0.2 to about 0.5 mm.

In an aspect, formation of a suitable spray is a critical operating parameter in ESI. Suitable solvent removable filters may be used to remove undesired solvents in the biological sample composition prior to being fed to the ESI. Generally high concentrations of electrolytes are avoided in samples fed to ESI.

The composition of materials of the means housing or holding the orifice and the orifice are compatible with the compositions of the biological sample to be processed through the orifice. Metallic and composition plastic compositions may be employed. In an aspect the orifice is a capillary or has a conical or capillary shape. In another aspect the orifice is cone shaped with the exterior converging from the proximate end to the distal end.

In an aspect, the biologic sample is forced through the orifice by application of air pressure to the sample at the proximate end of the orifice or the sample is forced through the orifice or capillary by the application of vacuum at the distal end of the orifice. The net result is that ions are suitably formed at atmospheric pressure and progress through the cone shaped orifice. In an aspect the orifice is a first vacuum stage and the ions undergo free jet expansion. A collector at the distal end of the orifice collects the ions and guides the ions to a tandem mass spectrometer (MS/MS).

The construction of a suitable vector can be achieved by any of the methods well-known in the art for the insertion of exogenous DNA into a vector. See Sambrook et al., 1989, Molecular Cloning, A Laboratory Manual Cold Spring Harbor Press, N.Y.; Rosenberg et al., Science 242:1575-1578 (1988); Wolff et al., Proc. Natl. Acad. Sci. USA 86:9011-9014 (1989). For Systemic administration with cationic liposomes, and administration in situ with viral vectors, see Caplen et al., Nature Med., 1:39-46 (1995); Zhu et al., Science 261:209-211 (1993); Berkner et al., Biotechniques, 6:616-629 (1988); Trapnell et al., Advanced Drug Delivery Rev., 12:185-199 (1993); Hodgson et al., BioTechnology 13:222 (1995).

While my discovery has been described in terms of various specific embodiments, those skilled in the art will recognize that my discovery can be practiced with modification.

1. A method for intentionally modulating the amount of fat in a living mammal having adipocyte cells which comprises administering a compound thereto having reactive selectivity to the metabolic regulatory system of the cells of the mammal to increase the fatty acid α oxidation of lipids in the adipocyte thereby reducing the amount of equivalents available for ATP synthesis whereby the amount of fat is reduced.

2. A method in accordance with claim 1, wherein the living mammal is a human.

3. A method in accordance with claim 1 wherein the compound is identified by administering a candidate compound to that living tissue and measuring alterations in fatty acid α oxidation flux and determining that the compound is a modulator if changes in fatty acid α oxidation occur if odd chain length fatty acid production appears, its kinetics are altered, or in the alternative, a net decrease in fat mass appears per given amount of caloric intake.

4. A method for modulating the amount of fat in a living mammal having adipocyte cells which comprises administering a compound thereto having a reactive selectivity to the metabolic regulatory systems which decreases the amount of α oxidation whereby small amounts of increased fat mass are obtained to decrease insulin requirements of diabetic patients and protect against atherosclerosis.

5. A method in accordance with claim 4, wherein the living mammal is a human and the amount of fat is reduced.

6. A method in accordance with claim 5, wherein the increase in α oxidation is mediated by a transcription-mediated increase in phytanoyl CoA α hydroxylase mRNA.

7. A method in accordance with claim 6, wherein the increase in phytanoly CoA αhydroxylase mRNA is followed by its translation and PAHX catalyzed α oxidation or other related chemically homologous reactions in the peroxisomal compartment of at least one cell in the mammal.

8. A method for modulating the energy dissipation in a living mammal whereby the energy dissipation is intentionally noninvasively redirected by the effective administration to the body of a compound having reactive selectivity enhanced to redirect the energy stores in that body to the metabolic regulatory system of the cell of that animal wherein the compound accelerates or promotes fatty acid alpha oxidation of the lipids in the adipocyte cells or other cells containing unwanted or toxic lipids.

9. A method in accordance with claim 8, wherein the living mammal is a human.

10. A method in accordance with claim 9 wherein compound causes a reduction in the amount of effective equivalents of carbons in FA for ATP synthesis by increasing α oxidation.

11. A method in accordance with claim 9 wherein the model is a murine.

12. A method in accordance with claim 10, wherein the model is a mammal.

13. A method in accordance with claim 12 wherein the mammal is a living human.

14. An animal model useful for identifying a compound which modulates the amount of fat in a living mammal which presents as a capable target for the compound mediating fatty acid α oxidation a selection of tissue having fatty acid α oxidation capability in the peroxisomal compartment of cells of that tissue, the model comprising a reconstituted enzyme system, cellular fraction (e.g peroxisomes), cell or living mammal

15. A method in accordance with claim 14, wherein the model is a murine.

16. A method for identifying a compound which modulates the amount of fat in a living mammal which presents itself as target for a compound mediating fatty acid α oxidation and a selection of tissue having fatty acid α oxidation capability in the peroxisomal compartment of cells of that tissue, which comprises administering a compound to that living tissue and measuring alterations in fatty acid α oxidation flux and determining that the compound is a modulator if changes in fatty acid α oxidation occur if odd chain length fatty acid production appears, its kinetics are altered, or in the alternative, a substantial net decrease in fat mass appears per given amount of caloric intake.

17. A method in accordance with claim 16, wherein the compound is determined to be a modulator if a substantial net decrease in fat mass appears per given amount of caloric intake.

18. A method for intentionally releasing fatty acid from its respective parent lipid (non polar or polar lipid) to the peroxisome for alpha oxidation and reduction of fat content by the activation of an endogenous adipocyte phospholipase or lipase to the metabolic regulatory system of cells of a living mammal, the adipocyte phospholipase selected from the group consisting of phospholipase gamma, phospholipase epsilon, phospholipase zeta and phospholipase eta and the subsequent alpha oxidation of the released fatty acid thereby reducing the lipid content and overweightness of the living mammal.

19. A method in accordance with claim 18 wherein the phospholipase is phospholipase gamma.

20. A method in accordance with claim 18 wherein the phospholipase is phospholipase epsilon.

21. A method in accordance with claim 18 wherein the phospholipase is phospholipase zeta.

22. A method in accordance with claim 18 wherein the phospholipase is phospholipase eta.
